(19) United States
(12) Patent Application Publication
(10) Pub. No.: US 2023/0251252 A1
(43) Pub. Date: Aug. 10, 2023

(54) DIGITAL SENSOR DEVICE FOR DETECTING AN ANALYTE IN A SAMPLE

(71) Applicant: digid GmbH, Mainz (DE)

(72) Inventors: Konstantin KLOPPSTECH, Mainz (DE); Constantin VON GERSDORFF, Mainz (DE); Nils KOENNE, Mainz (DE); Sanja RAMLJAK, Mainz (DE); Malte BARTENWERFER, Mainz (DE)

(21) Appl. No.: 18/133,096

(22) Filed: Apr. 11, 2023

Related U.S. Application Data
(63) Continuation of application No. PCT/EP2022/057648, filed on Mar. 23, 2022.

(30) Foreign Application Priority Data
Mar. 23, 2021 (DE) 102021107254.9

(51) Int. Cl. G01N 33/543; G01N 33/569; G01N 15/06

(52) U.S. Cl. CPC G01N 33/5438; G01N 33/56983; G01N 15/0606; G01N 2015/0065

(57) ABSTRACT

A sensor device is provided for detecting an incidence and/or a concentration and/or an amount of an analyte in a sample. The sensor device includes a sensor, connection electronics and a housing. The sensor converts chemical and/or biochemical information of an analyte in a sample into an electrical signal. The sensor includes a test cantilever that has a base and a deformable part, where a receptor layer for selective reception of the analyte is applied at least to the deformable part. The sensor also includes a reference cantilever that has a base and a deformable part, where a reference layer for selective non-reception of the analyte is applied to the deformable part.

### CROSS REFERENCE TO RELATED APPLICATIONS

This application is a continuation of PCT Application No. PCT/EP2022/057648, filed Mar. 23, 2022, which claims priority to German Patent Application No. 10 2021 107 254.9, filed Mar. 23, 2021, the entire contents of each of which are hereby incorporated by reference in their entirety.

### TECHNICAL FIELD

The present invention relates to a sensor device for detecting an analyte in a sample to derive a qualitative and/or quantitative statement regarding the presence of the analyte in the sample.

### BACKGROUND

Due to the increasing number of people across the world infected with the Sars-CoV2 virus, one of the main pillars for stemming the epidemic is the extensive testing of patients and the early tracking of infection chains.

Until now, no reliable and cost-effective point-of-care screening tests for the prompt diagnosis of a Sars-CoV2 viral infection are available at the treatment location. The most widely used test is based on a reverse transcriptase PCR method (Corman V M, Landt O, Kaiser M, et al. Detection of 2019 novel coronavirus (2019-nCoV) by real-time RT-PCR. Euro Surveill. 2020; 25(3):2000045. doi:10.2807/1560-7917.ES.2020.25.3.2000045), which requires a relatively high amount of effort due to the analysis in specialist diagnostic laboratories and an associated time outlay of up to three days between the sample being taken and the result being present with the medical staff and the patient. This delay causes uncertainty that lasts a relatively long time for the patient along with a significant delay both in the targeted treatment of the patient and in applying appropriate measures to stem the epidemic.

Existing point-of-care test methods are based on determining the anti-viral immune reaction by way of measuring IgG and IgM antibodies (Li Z, Yi Y, Luo X, et al. Development and Clinical Application of A Rapid IgM-IgG Combined Antibody Test for SARS-CoV-2 Infection Diagnosis [published online ahead of print, 2020 Feb. 27]. J Med Virol. 2020; 10.1002/jmv.25727. doi:10.1002/jmv.25727) by way of lateral flow-based immunochromatographic methods. Virus-specific antibodies are however able to be detected in plasma only 7 to 10 days following infection. In the case of SARS-CoV-2, patients are however already highly infectious in the first week following infection. This constitutes one of the main reasons for the rapid worldwide spread of the COVID-19 pandemic.

WO 2007/088018 A1 proposes spring elements for use in biosensors, such as for example in DNA analysis.

The deformation of cantilevers brought about by different surface stress is described for example in Rasmussen, P. A., Hansen, O., & Boisen, A. (2005). Cantilever surface stress sensors with single-crystalline silicon piezoresistors. Applied Physics Letters, 86(20), 203502. https://doi.org/10.1063/1.1900299.

### SUMMARY OF THE INVENTION

Accordingly, it is an object of the present invention to provide an improved sensor device for detecting an analyte in a sample.

In particular, a sensor device is provided for detecting an incidence and/or a concentration and/or an amount of an analyte in a sample, comprising a sensor, connection electronics and a housing, wherein the sensor is configured to convert chemical and/or biochemical information of the analyte into an electrical signal, comprising a test cantilever that has a base and a deformable part, wherein a receptor layer for selective reception of the analyte from the sample is applied at least to the deformable part, wherein the sensor comprises a reference cantilever that has a base and a deformable part, wherein a reference layer for selective no-reception on the analyte from the sample is applied to the deformable part.

The base of the test cantilever and/or the base of the reference cantilever may be formed as a rigid base. A rigid base is in this case understood that a deformation of the respective cantilever, that is to say of the test cantilever and/or of the reference cantilever, does not, or does not substantially, take place in relation to the deformable part of the respective cantilever. The rigid base is in this case for example connected to a substrate, supported by a substrate or machined out of the substrate. In contrast, the deformable part of the test cantilever and/or of the reference cantilever is not supported by the substrate, but rather formed as protruding beyond an edge of the substrate and being accordingly free.

The deformable part of the test cantilever and/or of the reference cantilever may for example be deflectable. The respective cantilever may in this case for example be deflected around a bending edge formed in a transition region between the base and the deflectable region. The bending edge is in this case for example the edge of the substrate along which the cantilever is divided into the base and the deformable part.

The deformation of the respective cantilever in its deformable part is however not limited to a deformation that rises or falls, and the cantilever may also be deformed in and of itself, being for example bulged or corrugated or distorted.

A sample in this case denotes a limited amount of a substance that has been taken from a larger amount of the substance, for instance from a reservoir, wherein the composition of the sample is representative of the composition of the substance in the reservoir and the corresponding incidence in the reservoir may accordingly be derived from the incidence of the substance in and the substance compositions of the sample.

For example, a sample may be a saliva sample, or a blood sample, or a swab, in particular a throat swab or a nose swab or a sinus swab, or extracted tissue. A sample in particular comprises any kind of biological sample, also including in particular samples from animals.

A sample may also be a non-biological sample, for example a sample of a chemical substance.

An analyte is in this case the substance the presence of which in the sample is intended to be detected in terms of quality and/or quantity or is intended to be detected by the sensor. The analyte may in particular be present directly in the sample, or be dissolved in the sample, or adhere to the sample or to part of the sample, in particular to a sample particle. The analyte may also undergo a chemical, biological and/or physical interaction with the sample, such that the analyte is able to be detected only indirectly via a corresponding interaction.

In particular, a sample form may be converted to another sample form, such that the analyte, or its incidence, is able to be detected in a simple and reliable manner. For example, a swab may be dissolved in a fluid such that the swab dissolved in the fluid is then the actual sample that is examined in terms of the analyte.

The analyte in the sample may also be chemically pre-treated, for example—if the analyte is a virus—by opening the viral envelope so as to arrive at nucleocapsid antigens. The analyte may also be labelled by such pretreatment in order to amplify the measured signal. For this purpose, antibodies may bind to antigens in order to generate the largest possible deformation on the cantilever system.

The sample then contains the chemical information and/or the biochemical information about the analyte. The chemical information may for example comprise the type of the analyte, the concentration of the analyte, the incidence of the analyte, the weight of the analyte, the reactivity of the analyte, the density of the analyte, etc. The biochemical information comprises the same properties as the chemical information, but these substances may for example arise due to biological processes. Reference is made in particular to biological information when the analyte has a particular influence on the biological cycle, for example metabolism or the immune system.

Preferably, a passive test transducer is arranged on the base of the test cantilever and an active test transducer is arranged on the deformable part of the test cantilever, and a passive reference transducer is arranged on the base of the reference cantilever and an active reference transducer is arranged on the deformable part of the reference cantilever, wherein the active and passive reference transducers and the active and passive test transducers are embodied and configured to output an electrical signal corresponding to the incidence and/or concentration and/or the amount of the analyte in the sample.

The chemical and/or biological information is converted into an electrical signal. This may mean that an electrical signal may be changed by or constructed from the chemical composition of the analyte. This may for example concern the conductivity of a circuit. For example, first biochemical information may be present when the circuit is conductive, and second biochemical information may be present when the circuit is not conductive.

In addition to the direct accessibility of the information, such as for example through conductivity, it is however also possible to derive the biochemical information through a physical and/or chemical process and/or an interaction.

For this purpose, the proposed sensor comprises a reference cantilever and a test cantilever. A cantilever is in this case a spring element that has a base and a deformable part. The base is accordingly a non-moving part of the cantilever, which is arranged on a substrate, in particular in a stationary manner. The deformable part of the cantilever is arranged on the base and protrudes beyond the substrate on which the base is arranged. The base and the cantilever may in particular be formed as one piece with one another. In other words, the deformable part of the cantilever is suspended on the base on one side. Since the deformable part protrudes beyond the substrate, the deformable part of the cantilever is able to bend, deflect and stretch. The spatial limit to which the cantilever is able to bend or the cantilever transitions from the base into the deformable part is called bending edge. The bending edge is usually an edge of the substrate when the cantilever protrudes beyond the substrate.

If the cantilever is bent, then this results in material stresses and forces in or on the material of the cantilever that can be measured. If such a material stress and/or force can be measured, bending of the cantilever may be derived therefrom.

The transducers have the purpose of determining or of measuring the deformation or the change in the surface stress of the cantilevers. The active transducers are arranged on the deformable parts of the cantilevers, whereas the passive transducers are arranged on the bases, for example the bases, of the cantilevers. The transducers may in particular be used to influence electrical properties of a circuit.

For example, a deformation or a change in the surface stress of the cantilever may lead to an increase in the resistance of a transducer, for example the active transducer, whereas no deformation or no alteration in the surface stress of the cantilever also does not alter the resistance of the transducer. This may be achieved for example via the embodiment of the transducers according to the principle of a strain gage, as a result of which a deformation of the respective cantilever is expressed by a change in length of the strain gage, applied thereto, of the transducer, and thus a deformation of the cantilever is able to be detected directly by a change in the resistance of the strain gage.

The chemical and/or biochemical information of the analyte can thus be detected by a deformation of the cantilever, a subsequent registration via a transducer, and finally via a change in an electrical property of a circuit.

A reference layer for selective non-reception of the analyte is arranged on the reference cantilever, while a receptor layer for reception of the analyte is applied to the test cantilever. A receptor layer is in this case a substance that is able to interact with the analyte. Interact in this case means that the analyte interacts chemically and/or biochemically and/or physically with the receptor layer. This in turn means that the receptor layer is selected specifically for each analyte.

Overall, the reference layer is ideally chemically very similar to the receptor layer, but preferably does not bind to any of the chemical species present in the sample.

In other words, the surfaces of the test cantilever and of the reference cantilever are preferably chemically identical with regard to possible interfering influences, but the reference cantilever does not react to any of the chemical species occurring in the sample with binding. The reference cantilever thus preferably behaves non-dynamically and does not bind to anything in the sample.

For example, a first analyte interacts only with a first receptor layer, while another analyte interacts only with another receptor layer. In contrast thereto, the reference layer is a substance with which the analyte explicitly does not interact. This in turn means that the reference layer is also analyte-specific and has to be selected accordingly.

The reference layer and the receptor layer advantageously have the common feature that the interaction with substances that are not the analyte is identically strong or identically weak in both layers. A substance that is not the analyte accordingly interacts just as strongly or just as weakly with the receptor layer as with the reference layer.

The selective reception of the analyte on the test cantilever has the effect that the test cantilever reacts sensitively to the analyte with a deformation and/or change in surface stress, and a signal is thereby generated by the transducer. The other substances in the sample that are not the analyte accordingly contribute only to background noise on the test cantilever.

In other words, due to the selective non-reception of the analyte on the reference layer, the interaction of the reference cantilever with the sample that comprises the analyte corresponds to the interaction of the test cantilever with the same sample that does not comprise the analyte.

In contrast, reference cantilevers according to the prior art do not have a receptor layer that reacts sensitively to the analyte. The reference cantilevers according to the prior art are also said to be “inert”.

Although effects such as turbulence in the sample and thermal drift of the sensor system may thereby be determined: However, in such a reference cantilever according to the prior art, the analyte may for example bind to the reference layer of the reference cantilever through non-specific binding. Thereby the analyte itself contributes to the background noise. Therefore, in the case of a sensor according to the prior art, there is a need for reference measurements in a reference sample, that is to say a sample without analytes. Only in this way, the effect of the non-specific binding of the substances that are not the analyte can be detected.

In the sensor proposed here, the selective non-reception of the analyte by the reference cantilever drastically simplifies the measuring method, since the reference cantilever is not sensitive to the analyte, and therefore the analyte also does not contribute to the background noise. Only the substances that are not the analyte contribute here to the background noise of the reference cantilever. The selective non-reception of the analyte on the reference cantilever may enable that the reference cantilever is exposed to the same turbulences, the same thermal drift and the same influence of all substances that are not the analyte as the test cantilever and also as in a reference fluid. The difference is however that the reference signal is determined directly in the sample fluid.

In other words, it is no longer necessary to calibrate the reference cantilever in a defined reference sample, but rather the measurement with test cantilever and reference cantilever may be performed directly in the sample to be analysed.

A reference cantilever with a reference layer and a test cantilever with a receptor layer in particular enables a more specific analysis of the analyte than just a reference cantilever without a receptor layer, since both the reference layer and the receptor layer have a specific interaction or non-interaction with the analyte.

Since the active transducers are arranged on the deformable parts of the cantilevers, it is possible, using the active transducers, to find a metric that corresponds to the strength of the interaction of the analyte with the deformable part. The passive transducers on the other hand are arranged on the bases of the cantilevers, such that the interaction is reduced to those interactions that do not primarily deform the deformable part.

The structure of the sensor with a reference cantilever and a test cantilever has the advantage that two measurements can be performed simultaneously in the sample, wherein the measurement of the reference cantilever can calibrate the measurement of the test cantilever. This makes it possible to reduce environmental influences, for instance chemical, thermal, mechanical, electrical and fluidic interfering influences, on the respective measurement, such that an incidence of the analyte is able to be derived from the comparison of the measurement on the test cantilever and on the reference cantilever.

In particular, it is also possible to optimize the geometry of the reference cantilever with regard to a specific interfering influence in order then to take this interfering influence into consideration in a targeted manner in the evaluation. It is additionally possible to determine different interfering influences with different reference cantilevers.

In other words, what is also proposed is a structure of the sensor with a test cantilever and at least two reference cantilevers, wherein the different reference cantilevers are then preferably optimized for different interfering influences.

The selective reception of the analyte, preferably of the virus or of the antigen, by the receptor layer and the selective non-reception of the analyte, preferably of the virus, by the reference layer may cause a relative deflection of the test cantilever with respect to the reference cantilever, wherein the incidence of the analyte, preferably of the virus, is inferred, preferably the magnitude of the incidence is inferred, by comparing the forces detected by the transducers.

The transducers may be embodied and configured to ascertain deformations of the deformable parts of the test cantilevers and of the reference cantilever, preferably to detect the forces exerted on each base and the deformable parts of the test and reference cantilevers in the deformation.

If a sensor, consisting of a reference cantilever and a test cantilever with the reference layer and the receptor layer, respectively, is exposed to a sample that comprises an analyte, then the interaction may consist in the analyte binding to the receptor layer.

The binding of the analyte to the receptor layer changes the surface stress of that side of the test cantilever covered by the receptor layer, which leads to a force on the test cantilever, whereas no force acts on the reference cantilever due to the analyte. The force on the test cantilever for example increases more quickly the larger the concentration of the analyte in the sample or the more quickly the analyte covers the surface of the cantilever. A possible maximum force for the respective embodiment is achieved when the cantilever is fully covered.

This interaction may bring about a deformation in the deformable part of the test cantilever, whereas the deformable part of the reference cantilever is not bent.

The reason for the deflection of the cantilever is the change in the surface stress caused by the interaction with the analyte. The change in the surface stress leads to the upper (or lower) surface of the cantilever expanding or contracting. The different stretching or contraction on the upper and lower side brings about an internal force or material stress in the material that leads to the deformation.

These forces or material stresses and in particular stretches or contractions may ultimately be detected by the transducer, wherein stretches or contractions of different magnitudes are detected by the transducer through voltages of different magnitudes.

The force to be detected may be a bending force and/or a stretching force and/or a contraction force and/or a shear force and/or be based on the modulus of elasticity of the reference cantilevers and test cantilevers.

A bending force may change the geometry of the cantilever, in particular impress a curvature on the cantilever that differs from the unstressed cantilever. Such a curvature may lead to the occurrence of bending moments or stretchings and thus to bending stresses that are able to be determined using an appropriate transducer.

The change in the surface stress and the resulting stretching force may in particular change a length of the cantilever in this region. The stretching of the upper surface may in particular be different from the lower surface of the cantilever. The stretching of the upper surface may in particular take place parallel to the base of the cantilever (what is known as a transverse stretching) or perpendicular to the base of the cantilever (what is known as a longitudinal stretching). The magnitude of the stretching is in this case strongly dependent on the geometry of the cantilevers and on the other layers provided on the surfaces, for example the electrodes, such that it is possible to achieve optimum detection of the analyte by optimizing orientation and cantilever geometry. The respective change in length may be different depending on the direction of the crystal lattice of the cantilever.

Preferably, the relative deformation and/or the relative change in the surface stress runs in a transverse direction (that is to say in the direction of the transverse stretching) of the test cantilever and/or of the reference cantilever, wherein the transverse direction runs parallel to the base of the test cantilever and/or of the reference cantilever, wherein the active and the passive test transducer and/or the active and the passive reference transducer are preferably oriented in the transverse direction.

The relative deformation and/or the relative change in the surface stress furthermore preferably runs in a longitudinal direction (that is to say in the direction of the longitudinal stretching) of the test cantilever and/or of the reference cantilever, wherein the longitudinal direction runs parallel to the base of the test cantilever and/or of the reference cantilever, wherein the active and the passive test transducer and/or the active and the passive reference transducer are preferably oriented in the longitudinal direction.

If the stretching force is different on the upper surface and the lower surface, the acting force is also referred to as shear force.

In the case of a deflected cantilever, a bending force is active since a curvature is impressed on the cantilever. The upper surface of the cantilever is thereby stretched and this stretching is in particular larger than on the lower surface of the cantilever, such that overall a shear force acts on the cantilever.

The abovementioned forces are all based on what is known as the modulus of elasticity of the cantilever. The modulus of elasticity of the cantilever is a material constant that is specific to the material used for the cantilever. Selecting the material or the material composition or treating the material makes it possible to set the modulus of elasticity in a particular range, such that the effect to be measured can be optimized for the respectively configured transducer. On the other hand, it is of course also possible to adapt the transducers to the existing modulus of elasticity of the material and to optimize their sensitivity.

The cantilevers may preferably be so-called bimaterial cantilevers, for example cantilevers made of a gold layer and a silicon nitride layer. A bimaterial cantilever includes of material layers that together have a defined state of stress. For example, the state may be stress-free, such that the intrinsic mechanical stresses are minimal. It may however also be the case that a bimaterial cantilever is prestressed, such that the cantilever reacts particularly sensitively to a change in surface stress. It may however also be the case that a homogeneous cantilever is coated differently on the upper side and the lower side in order to imitate the described bimaterial effect.

Comparing the deformations and/or forces detected by the transducers makes it possible to derive an impact, caused by the selective reception of the analyte, on the test cantilever, and thus the incidence of said analyte. The magnitude of the incidence may preferably be derived.

The passive transducers on the bases of the cantilevers in particular detect only effects that are primarily not a deflection, since the bases are coupled fixedly to the substrate.

The measurement signals from the passive transducers thus yield a basic signal that is specific to the respective cantilever.

For example, the base of the reference cantilever, due to the influence of the environmental conditions, may bring about a first electrical state of the passive reference transducer, while the interaction of the test cantilever with the sample brings about a second electrical state of the passive test transducer.

In contrast, the active transducers on the deformable parts of the cantilevers indicate an extent of the deformation or acting force and thus also indirectly an extent of the interaction of the reference layer or receptor layer with the analyte and the other substances in the sample.

For example, the reference cantilever may be bent by a first amount as a result of interacting with the sample, such that the deflection brings about a third electrical state in the active reference transducer, whereas the test cantilever is bent by a second amount as a result of interacting with the sample and is bent by the third amount as a result of additionally interacting with the analyte in the sample, which brings about a fourth electrical state in the active test transducer.

Comparing the electrical states of the passive and active transducers indicates a measure of the deformation of the cantilevers, wherein the electrical signal from the active transducers is calibrated to the basic signal from the passive transducers on the base. Simultaneously, a comparison of the active transducers, or the measured signals from the active transducers, gives a measure of the difference between the deformation of the cantilevers. It is thereby possible to infer a specific influence of an analyte on the test cantilever.

The structure with four transducers has the advantage that such local calibration of the sensor is possible at the location of the influence of the sample and of the analyte.

The deformable parts of the reference and test cantilevers may have identical geometric dimensions, wherein preferably the width of the deformable part of the reference and test cantilevers corresponds to the length of the deformable part of the reference and test cantilevers, wherein the deformable parts of the reference and test cantilevers are particularly preferably less than 100 μm wide, less than 100 μm long and less than 1 μm thick, in particular 50 μm wide, 50 μm long and 0.3 μm thick.

This makes it possible to produce a particularly large force on the active transducers through the deformation of the cantilevers.

The bases of the reference and test cantilever may be arranged on the same overall base. The test cantilever and the reference cantilever in particular have a common base.

This makes it possible to achieve a situation whereby the passive transducers operate on a similar basic level, for example influences that would be specific for the separate bases are reduced. This may lead to an increased measurement accuracy.

The reference and test cantilevers may in particular thereby be arranged particularly close to one another, for example less than the width of one cantilever.

This makes it possible to achieve a situation whereby identical interfering influences act on the cantilevers, which are caused for example by a temperature difference in the sample, in particular by convection or another fluid dynamic.

This furthermore also makes it possible to achieve a situation whereby multiple cantilevers can be produced from one (silicon) wafer. This enables a cost-effective production of the sensor.

The distance of the cantilevers may furthermore be optimized towards the production limit. The production limit is typically defined by the spotting distance, wherein the spotting distance is a metric that is relevant when producing the reference layer and receptor layer, see below.

The bases of the reference and test cantilever may be formed in one piece with one another.

This makes it possible to achieve a situation whereby the base-specific influences are further reduced, such that an increased measurement accuracy is achieved. This furthermore makes it possible to achieve simpler production of the cantilever pair or of a multiplicity of cantilever pairs.

The deformable parts of the reference and test cantilevers may thus in particular have identical geometric dimensions, wherein the width of the deformable parts of the reference and test cantilevers corresponds to the length of the deformable parts of the reference and test cantilevers, the bases of the reference and test cantilevers are arranged on the same overall base and the bases are preferably formed in one piece with one another.

The reference and test cantilevers may comprise Si3N4, SiO2, Si3N4/SiO2, SiC, Si or comprise of Si or comprise a polymer.

By using silicon-based reference and test cantilevers is possible to use production methods known from the semiconductor industry, thereby making it possible to produce sensors according to the invention on a large industrial scale. Polymers may likewise be produced on a large industrial scale and have the advantage that their material properties can be predetermined to a large extent.

The transducers may have identical intrinsic physical properties, wherein the transducers are configured to adapt their electrical properties, preferably the electrical resistance or another value proportional to the k-value, in accordance with the forces acting on the reference and test cantilevers.

The k-value, also called gauge factor, is the proportionality constant between the stretching of the transducer and its change in resistance:

$$\frac{Δ R}{R} = k \frac{Δ L}{L} ,$$

wherein ΔR is the change in resistance of the transducer, R is the resistance of the transducer when the cantilever is not bent, ΔL is the change in length of the transducer and L is the length of the transducer when the cantilever is not bent. Any other measured variables proportional to the k-value or to the resistance, such as for example conductivity, may in particular also be measured.

Identical intrinsic physical properties in this case comprise those properties that are responsible for the measurement properties of the transducer on a cantilever. This concerns in particular a voltage that drops across the transducer, that is to say the resistance or the conductivity of the transducer. The resistance depends in particular on the geometry of the transducer, such that, in the case of uniform conductivity of the various transducers, the geometry of the transducers accordingly has to be identical.

The physical properties however also relate to the way in which the measured signal changes in response to an acting force. Each transducer should in particular react identically to an identical exerted force or deformation of the cantilever, such that no non-linear deviations may occur between the various transducers.

The intrinsic physical properties are defined in particular by the nanostructure of the transducers. The nanostructures are preferably identical for all transducers, such that identical geometric configurations yield identical physical properties.

This may in particular be achieved by virtue of the metal particle grain sizes of the transducers being as small as possible.

A reliable production process for the transducers thus makes it possible to ensure that all transducers react identically to a force, such that deviations between the various measured forces are based only on the external action on the cantilevers and do not depend on the intrinsic physical properties.

The electrical properties of the transducers may in particular ultimately be used to derive the bending states of the individual reference and test cantilevers, wherein in particular the incidence of the analyte received selectively by the receptor layer may be derived. The transducers, which detect an interaction with the analyte indirectly via the cantilever, vary their measurement properties in accordance with the exerted forces.

The distance between the active reference transducer or test transducer and the passive reference transducer or test transducer, respectively, may be less than 100 μm, wherein the transducers may lie against the bending edge.

Since the transducers are placed as close as possible to one another, spatial influences on the transducers stemming from the sample are reduced. If for example the incidence of the analyte in the sample is subject to a particular concentration gradient, it is advantageous to perform the measurements as far as possible at a point of the gradient.

The smallest possible distance is achieved when the transducers lie against the bending edge. The bending edge is in this case the edge of the substrate along which the cantilever is divided into the base and the deformable part. For example, the active transducers may lie against the bending edge with their lower edge, while the passive transducers may lie against the bending edge with their upper edge.

The optimum distance between the active transducers and the bending edge may in particular depend on the precise geometric shape of the sensor. The distance to the bending edge may accordingly be selected such that a surface stretching generates a maximum change in the electronic state of the transducer.

The optimum distance between the passive transducers and the bending edge is achieved in particular when the deflection of the test cantilever achieves the smallest possible change in the electronic state of the test transducer.

The active and passive transducers should however in particular be arranged so close to one another that they are able to be written together easily and quickly in a single step in a production process, for example in a scanning electron microscope-based production process, without a mechanical movement of an XYZ advancement device having to displace the wafer. This in particular enables considerably faster and more accurate and also inexpensive production of the sensors.

The orientation of the transducers in particular defines whether a longitudinal or a transverse stretching of the cantilevers is measured. When a longitudinal axis of the transducer runs parallel to the base, a transverse stretching of the cantilever is preferably measured. When a longitudinal axis of the transducer is oriented perpendicular to the base, the longitudinal stretching of the cantilever is preferably measured. It is therefore in particular also possible to form rectangular, square, round or oval transducers in order to adapt the sensitivity of the transducer to the cantilever geometry.

The reference and test cantilevers, and the active and passive reference and test transducers, may be arranged mirror-symmetrically to one another.

The mirror symmetry may relate in particular to a mirror axis that is arranged between the reference transducer and the test transducer.

A mirror-symmetric structure makes it possible to reduce influences, for example of voltages, on the transducers, or at least to channel them symmetrically to one another. The measurement accuracy and susceptibility to interference may thereby be improved.

The sensor may have electrodes, preferably have four electrodes that are configured to electrically connect the transducers.

An electrode is in this case a conductive layer, for example made of gold, or a wire or cable that can create, from a connection end of the transducer, an electrically conductive connection to an external device, such as for example a current or voltage source or to a corresponding measuring device.

Any conductive connection between the transducer and the external device may be understood in principle to be an electrode. However, in particular the part of the electrical connection that is formed on the sensor is considered here to be an electrode.

The sensor is typically electrically connected to an external source or a measuring device. In this case, an electrical connection plug is connected by a cable or a wire to a so-called bonding pad, for example by virtue of the wire being fixed by ultrasonically welding. An electrical connection then leads directly from the bonding pad to the transducer. The electrical isopotential surface between the transducer and the bonding pad is hereinafter called electrode.

The electrode serves to electrically connect the transducers and in particular to provide the possibility of routing the electrical signals from the sensor to a measuring device.

The electrodes may in particular be at different electrical potentials and interact with one another thereby. In order to minimize this mutual influencing of the electric currents and voltages in the electrodes, it is therefore advantageous for the electrodes likewise to have a symmetrical form such that the respective interference is at least distributed uniformly among the overall system. This may in particular be achieved by using an even number of electrodes or using only four electrodes in the case of four transducers.

Due to the form of the electrode geometry, it is therefore possible for the basic signal level that arises at the electrodes due to any potential differences to be less than 1.1 V, such that it is not necessary to electrically encapsulate the electrodes. Electrical encapsulation may in this case be understood to mean for example electrical insulation or covering or shielding of the electrodes and of the bonding wires. The production process may thereby be simplified, and measurement accuracy is improved.

The distance between the electrodes may be minimal.

Minimal is in this case the distance when the electrodes are not touching, that is to say are not conductively connected to one another. In other words, the conductance between the electrodes is significantly lower than the conductance of the transducers.

Since the distance between the electrodes is minimal, more electrodes can be placed on a wafer, thereby enabling a cost-effective production process. It is thereby however also possible in particular to reduce the size of the transducers, such that the influence of non-uniform environmental conditions on the transducers is able to be further reduced.

The transducers may be electrically interconnected in a full bridge, wherein the full bridge is configured to build up a transverse bridge voltage based on the electrical properties of the transducers, in particular in the event of an asymmetric change in the electrical properties of the transducers.

A full bridge is in this case a measuring apparatus for measuring electrical resistances or small changes in resistance. A full bridge is also known by the names Wheatstone measuring bridge or H-bridge or symmetrical full bridge or thermo-symmetrical full bridge.

For example, the active and passive transducers of the reference and test cantilevers are interconnected to form a full bridge by connecting a contact of the active transducers each to a common potential via a first electrode. A respective connection of the contacts of the passive transducers is furthermore set to a common potential via a third electrode. A voltage (DC or AC voltage) may be applied via this first and third electrode, wherein the combination of the active transducers or the passive transducers in each case acts as a voltage divider in accordance with the resistances of the respective transducers.

In addition, on each cantilever, the further contact of the active transducer is connected to the further contact of the passive transducer via a second electrode in the case of the test cantilever or fourth electrode in the case of the reference cantilever. A transverse bridge voltage is accordingly built up across the second and fourth electrode if the ratio of the resistances of the active transducer to the passive transducer of the reference cantilever is not the same as the ratio of the resistances of the active transducer to the passive transducer of the test cantilever.

In the basic state of the full bridge of the sensor, the transverse bridge voltage is ideally equal to zero, since no force or an identical force on all of the transducers is involved. This basic state is preferably already set in the production process, such that only a slight offset voltage that may be compensated for via a measurement structure is established between the electrodes.

Proceeding from this basic state of the full bridge, asymmetric force changes may then preferably be detected. If for example the active test transducer of the test cantilever reacts to an exerted force with a change of its electrical property, respectively with a change in its electrical resistance, then the ratio of the resistances in the full bridge is no longer balanced, such that a transverse bridge voltage builds up. The built-up transverse bridge voltage may ultimately be detected using a measuring device.

In particular, no transverse bridge voltage builds up when the exerted force on the active transducers of the test cantilever and of the reference cantilever is identical. This is then however a non-specific exerted force that does not stem from a specific interaction with the test cantilever. In particular, no transverse bridge voltage builds up when the exerted force on the passive transducers of the test cantilever and of the reference cantilever is identical.

By realizing the system as a full bridge, the active reference transducer of the reference cantilever is used to calibrate the active test transducer of the test cantilever. The passive transducers enable a calibration to the basic state of the full bridge, on the one hand, and a deflection of the deformable parts of the cantilevers may be derived by comparing the active and passive transducers, on the other hand.

The sensor may comprise a transverse bridge voltage detector that is configured to detect the transverse bridge voltage of the full bridge, wherein the detected transverse bridge voltage is used to derive the incidence of the analyte selectively received by the receptor layer, preferably to derive the magnitude of the incidence.

A transverse bridge voltage detector may in particular be any detector that is capable of detecting a voltage. For example, this may be a measuring resistor, or a signal transmitter or a measuring device that indicates the voltage or another type of detector that generates an output signal by detecting a voltage.

The transverse bridge voltage detector may be configured to generate a single output value, such that only the occurrence of a transverse bridge voltage is indicated. The occurrence of a transverse bridge voltage may in particular be taken as a basis for inferring that an analyte in a certain minimum concentration has interacted with the receptor layer of the test cantilever, and as a result the electrical properties of the transducers, or at least the electrical property of the active transducer of the test cantilever, have changed.

A transverse bridge voltage detector may however also indicate various output values that preferably have a simple functional relationship with the transverse bridge voltage. For example, this may mean that the output value of the transverse bridge voltage detector rises if the transverse bridge voltage rises. However, this may also mean that the output value from the transverse bridge voltage detector drops if the transverse bridge voltage rises. It is particularly advantageous to infer an unambiguous value of the transverse bridge voltage from the output value from the detector. In other words, it is preferable for the output value from the transverse bridge voltage detector to follow a bijective function of the transverse bridge voltage.

The change in the bridge voltage is ideally expressed as a ratiometric change in relation to a defined, that is to say measured, supply voltage. For example, a drift in the supply voltage then does not influence the measured signal.

The various output values do not have to be restricted to the amplitude of the signal, but rather may be restricted to the temporal occurrence of the output value.

For example, the transverse bridge voltage detector may output one pulse per time interval in the case of a first voltage, whereas the transverse bridge voltage detector outputs four pulses per time interval in the case of a second voltage. The occurrence of the pulses may thus be used to indicate the strength of the transverse bridge voltage. The output value may in particular thus be coded.

The electrical properties of the transducers may be output via an A/D converter and an A/D converter logic unit may be configured to provide a differential measurement and/or an absolute measurement of the bending states.

The transverse bridge voltage detector may in particular be embodied in the form of an A/D converter, wherein an A/D converter is a converter electronics system that generates a digital signal from an analog signal. For this purpose, for example, the strength of the measured signal is sampled pointwise with a certain periodicity by the A/D converter, and the measured voltage is converted into a digital value.

The A/D converter may in particular comprise an A/D converter logic unit, wherein the A/D converter logic unit is able to be put into different operating modes by adjusting the inner circuitry, in particular through software modifications. The various operating modes may be used to tap off various voltages (in particular AC voltages and/or DC voltages) and measured signals from the electrode circuit.

For example, the A/D converter may have what is known as a differential measuring mode in which only the change in bending state between the reference cantilever and the test cantilever is detected. In this differential measuring mode, the transverse bridge voltage is in particular tapped off such that a change in the bending states of the cantilevers is detected in the form of an occurring transverse bridge voltage. The differential measuring mode is the preferred measuring mode for detecting binding of an analyte to the receptor layer.

However, it is also possible to operate the A/D converter in what is known as an absolute measuring mode, in which the individual transducers are accessed directly via the electrodes (single ends mode). This allows to measure the individual transducer resistances, for example for quality assurance purposes, or else in order to characterize the full bridge. It is thereby furthermore possible to detect the absolute bending states of the cantilevers.

Overall, an A/D converter may convert the transverse bridge voltage into a digital signal, wherein the A/D converter can be operated in a differential measuring mode and/or in an absolute measuring mode using an A/D converter logic unit.

In particular in this structure, only one A/D converter is required, such that the production process is able to be implemented inexpensively.

Due to the stable and balanced full bridge, it is additionally also possible to move the A/D converter far away from the actual transducers and cantilevers, such that for example waste heat from the A/D converter does not influence the measurement result.

The sensor may be formed on a chip.

This may mean that the sensor is produced on a semiconductor structure that permits further data processing of the transverse bridge voltage or of the output value from the A/D converter logic unit. A chip may also be understood to mean in particular what is known as a system-on-a-chip, wherein all of the functional units of the measurement system are formed integrally on a single electronic component.

However, it should be taken into consideration that the production process for the sensor may comprise gold, which may impair the production of an A/D converter logic unit using CMOS semiconductor techniques.

A multiplicity of cantilever pairs may be arranged on a chip, wherein an A/D converter logic unit may be configured to provide signal multiplexing of the measured signals.

A cantilever pair comprises in each case a reference cantilever and a test cantilever. A multiplicity of such cantilever pairs along with active and passive transducers may be arranged on a chip and each be read in turn by an A/D converter logic unit.

It may also in particular be the case that a first cantilever pair reacts specifically to a first analyte and a second cantilever pair reacts to a second analyte, such that different analytes are able to be detected by a sensor.

A multiplicity of cantilever pairs may however also comprise a first number of test cantilevers and a second number of reference cantilevers. By way of example, the various reference cantilevers may detect different interfering influences in a particularly sensitive manner, these together delivering the reference for the number of test cantilevers.

The cantilever pairs may be operated simultaneously using an appropriate A/D converter logic unit. It is thereby possible to detect a large number of different analytes using different receptor layers and reference layers, on the one hand. On the other hand, however, it is also possible to establish a statistical statement about the significance of the measured transverse bridge voltages through identical receptor layers and reference layers.

The upper surfaces of the reference and test cantilevers may be activated by an activation layer, wherein the activation layer is configured, in the event of an exerted force on the reference and test cantilevers, to provide a larger surface stretching in comparison with the non-activated lower surface of the reference and test cantilevers, and wherein the activation layer comprises gold or other chemically inert materials.

Activating the upper surface may mean that an adhesion promoter for a further layer is provided by applying an activation layer. The reason for this may be that the base material of the cantilever for example does not bind to the further layer, in particular the reference layer.

The activation layer may in particular comprise gold, or consist entirely of gold.

The full surface of the cantilevers is preferably coated with gold, since the receptor layer is preferably constructed on the gold layer. Accordingly, an expansive layer containing the activation layer may also coat a larger surface containing the receptor layer, thereby giving a large detector surface for the analyte. Due to the large detector surface for the analyte, this in turn results in a particularly large deformation of the cantilever, such that sensitive detection of the incidence of the analyte is possible.

Since the upper surface has an activation layer, the structure of the cantilevers is in particular non-homogenous or asymmetric in terms of height, but rather consists of layers. The elasticity of the cantilever may thereby be influenced significantly, such that a larger surface stretching arises on the upper surface upon a deformation of the cantilevers, which in turn leads to a larger measured signal.

The coating of the cantilevers with gold may likewise be used, due to the good conductivity, to form electrodes for the transducers. For this reason, the distance between the electrodes may also be minimized, since the smallest possible surface of the cantilever is thus not coated with gold. The detector surface may accordingly be selected to be large.

The activation layer may in particular also consist of a chromium-gold alloy, since the mechanical properties of the cantilever are influenced less thereby. Admixing chromium in particular achieves homogeneity of the crystallites of the gold layer, such that it is possible to avoid any interfering anisotropic effects through the crystal lattice of a hypothetically crystalline layer.

The lower surfaces of the reference and test cantilevers may be passivated by a passivation layer, wherein the passivation layer is configured to minimize non-specific protein adhesion to the reference and test cantilevers, and wherein the passivation layer comprises trimethoxysilane and/or a blocking substance.

Unlike an activation layer, a passivation layer is a layer that is intended to minimize or prevent any interaction between the cantilever and another material. This means that, during the production of the receptor layer, this binds only to the upper surface of the cantilever and not to the lower surface of the cantilever. The binding of the receptor layer to an analyte thereby makes it possible to achieve a larger surface stress on the upper surface. This also increases the asymmetry of the layer structure, which may lead to improved stretching properties for the signal detection.

Particularly suitable for the passivation of the lower surface are the materials trimethoxysilane, and so-called blocking layers. This passivation layer minimizes a so-called non-specific protein adhesion. A protein adhesion is the adhesion of a protein to the surface. Non-specific adhesion of a protein or of a substance in general to the cantilevers may lead to distortions of the measurement result, since these non-specific substances likewise interact with the cantilevers. Preventing this non-specific adhesion increases the relative influence of the desired specific adhesion or interaction of the analyte with the cantilever relative to the basic state of the cantilever.

However, it is also possible for a passivation layer also to bind the analyte, but in such a way that the resulting surface stress is opposed to the surface stress of the activation layer. This makes it possible to achieve larger deformation of the cantilevers.

The so-called blocking layer may in particular be adapted to the respective analyte under investigation, in order to define a measurement window for the analyte. The blocking layer is in this case applied in a so-called spotting process or washing process.

In the washing process, a so-called “sealer” protects the hydrate envelope of the detector proteins during drying and thus makes them storable. The sealer either is incorporated into a matrix in soluble form, such that it is soluble for a sample fluid such as water. The sealer additionally has a certain layer thickness, such that the cantilevers are mechanically stabilized, which increases protection during storage of the cantilevers. A sealer may for example contain sugar. Sugar crystals are hydrophilic and therefore protect the hydrate envelope of the proteins. What is known as reconstitution of the proteins, in which the dried proteins are reactivated in the measuring fluid, is thus possible.

In the case of spotting of the receptor proteins, so-called “buffers” are used in order to enable reconstitution of the proteins in the sample fluid. In this case too, the storability of the sensors is increased by drying.

The active and the passive cantilever may have an identical chemical structure.

This results in that the measured signal, in particular in the case of a differential measurement of transverse bridge voltage, is based only on the influence of the analyte on the cantilevers and is not generated by further properties of the cantilevers.

Chemical identity in particular refers to the fact that the cantilevers are changed and adapted to the extent that they differ only in terms of their binding properties or interaction properties with respect to the analyte to be measured. An interaction that is as far as possible identical, or an interaction that is as low as possible, is intended to be achieved for all other substances.

For this purpose, the reference cantilever and the test cantilever have an identical layer structure that differs only in that a receptor layer is applied to the test cantilever and a reference layer is applied to the reference cantilever. Chemical identity thus in particular means that the two cantilevers differ only in terms of the reference layer or test layer.

On the whole, the overall layer structure of the cantilevers as described above may also be inverted. This means that the reference and receptor layers, instead of being applied to the upper surface, may also be applied to the lower surface of the cantilevers. For example, the receptor layer may also be arranged on the lower side of the cantilever.

In order for the cantilever to deform, any chemical bonding to the cantilever should ideally take place on one side. When the analyte binds to the upper side, there should not be any non-specific binding to the lower side of the cantilever, since otherwise the surface stress that results from the chemical binding of the analyte may be compensated for by the non-specific chemical binding to the lower side of the cantilever.

In other words, the chemical binding to the upper side and the lower side must be at least asymmetric in order to achieve a deformation. Stronger binding to the upper side than to the lower side or stronger binding to the lower side than to the upper side accordingly leads to a measurable deformation of the test cantilever.

The reference and test cantilevers may have a further layer that comprises a self-assembling monolayer.

A self-assembling monolayer may in particular reduce unevennesses on the gold surface, such that a uniform coating of the cantilevers with the receptor layer or reference layer is possible. The homogeneous surface properties of the cantilevers thus ultimately make it possible to improve the binding properties of the receptor layer and of the analyte.

The receptor layer may comprise antibodies for an antigen and the reference layer may comprise an antigen-specific isotype control antibody according to the antibody of the receptor layer.

Antibodies are proteins that are produced by body cells as a reaction product to antigens. Antibodies are typically used by the human immune system to bind to the antigens of viruses, such that the viruses are marked and an outbreak of a viral infection is able to be avoided by the immune system. It may in particular be the case that an antibody binds to different antigens, such that the specificity of the antibody is lowered.

An isotype control antibody in contrast thereto binds precisely not to the antigen of a virus, such that, in the event of the simultaneous presence of binding of the antibody to the antigen and of non-binding of the isotype control antibody to the antigen with high specificity, the presence of a particular virus or antigen of a virus may be inferred.

The antibody of an antigen may be part of the receptor layer of the test cantilever, while the isotype control antibody of the antigen may be part of the reference layer. This has the advantage that a deflection of the test cantilever can be confirmed simultaneously by the non-deflection of the reference cantilever.

The reference and receptor layer of the cantilevers may additionally have a so-called protein A for better adhesion of the antibodies, which binds covalently to the self-assembling monolayer.

In other words, a passivation layer may be applied to the lower surfaces of the reference and test cantilevers, an activation may be applied to the upper surfaces of the reference and test cantilevers, a self-assembling monolayer may preferably be applied to the activation layer, and a reference layer or receptor layer may preferably be applied to the self-assembling monolayer of the reference or test cantilever, wherein the receptor layer comprises antibodies for an antigen and the reference layer comprises an antigen-specific isotype control antibody according to the antibody of the receptor layer.

The layers may be produced in a dipping/spotting process, wherein the spotting may preferably be performed by way of commercially available machines. In this case, droplets of the respective layer are deposited on the cantilever, so as to spatially delimit the functionalization, which in particular enables inexpensive and independent coating of the cantilevers. The very small drops are prevented from drying by appropriately controlling the environmental parameters, such as temperature, air humidity and dew point. The lower sides of the cantilevers are not activated in this case, such that the antibodies that are used merely come into contact with the upper surface of the cantilever. The layers are then dried, such that a higher or lower temperature has little or preferably no influence on the antibodies. This allows a long storability, in particular in an inert gas. The protein layers are in particular applied after the transducers have been applied, but before the sensors are singulated from the wafer. The receptor layer may comprise Sars-CoV2 antibodies and the reference layer may comprise Sars-CoV2-specific isotype control antibodies.

The Sars-CoV2 antibody preferably binds to the Si or N antigen of the Sars-CoV2 virus.

The antibody is monoclonal, sequence-accurate and has high specificity with respect to the Sars-CoV2 antigen. The antibody may in particular be produced through what is known as the phage display method. The Sars-CoV2-specific isotype protocol antibody on the other hand may be ultra-high-specific to the corresponding antigen, but otherwise identical to the active antibody.

This enables for example rapid detection of the Sars-CoV2 antibody. The electrical measurement and the accumulation of the antibodies on the test cantilever in particular gives a rapid test method that, due to the comparison with the non-accumulation on the reference cantilever, additionally has high specificity.

The receptor layer may generally provide molecule-specific binding forces and the reference layer provides molecule-specific no binding forces. It is thereby possible to demonstrate a particular molecular species.

The receptor layer may comprise single-strand DNA (ssDNA) and/or other DNA fragments that are able to bind specifically to DNA fragments in the sample. The reference layer may comprise single-strand DNA and/or other DNA fragments that do not bind to any chemical and/or biochemical and/or physical species in the sample, but correspond to the receptor layer in terms of characteristic parameters (for example chain length, chemical structure).

The receptor layer may comprise single-strand RNA and/or other RNA fragments that are able to bind specifically to RNA fragments in the sample. The reference layer may comprise single-strand RNA and/or other RNA fragments that do not bind to any chemical and/or biochemical and/or physical species in the sample, but correspond to the receptor layer in terms of characteristic parameters (for example chain length, chemical structure). It is thereby possible to demonstrate a particular DNA or RNA and their fragments and/or other oligonucleotides.

The receptor layer may comprise antibodies and/or other and/or further proteins that are able to specifically bind target proteins and the reference layer may accordingly comprise specific isotype control antibodies and/or further proteins that do not bind to any chemical and/or biochemical and/or physical species in the sample.

The receptor layer may comprise scFv antibody fragments and the reference layer may comprise scFv antibody fragment-specific isotype control antibodies. An scFv antibody is an artificially produced antibody fragment. Since an antibody may be broken down into multiple fragments, the reactivity of the sensor to a low sample concentration may be increased.

The receptor layer and the reference layer may comprise hydrogels.

Hydrogels are molecular matrices that are able to bind water very well and that swell up to a great extent upon contact with water. By chemically modifying the hydrogels, in particular the matrix, it is possible to bring about a strong reaction of the hydrogel to the presence of proteins or other analytes, such that the mechanical deformation of the cantilever is multiplied. It is thus in particular also possible to perform a pH-sensitive measurement of the analyte.

The specific properties of the connection electronics are described below.

The connection electronics may comprise a printable circuit board that is configured to ensure the electrical communication between a connection socket and the sensor.

A printable circuit board is a circuit board that serves as carrier for electronic components. The circuit board is in particular tasked with providing a conductive connection between electronic components. The material of the printable circuit board is compatible with the desired application. For example, the material is fluid tight and/or has no non-specific protein adhesion. For example, the material also does not output any substances that interfere with the process of the analyte binding to the receptor layer.

An electronic component is in particular also a so-called connection socket, which provides a physical interface between the electronics of the printable circuit board and external voltage sources and measuring devices. The connection socket has microscopic extensions and accordingly has poor mechanical compatibility with the chip or the transducer in direct terms. Such a circuit board however easily makes it possible to connect the sensor to a connection socket.

The electrodes of the sensor, as already described, may be connected via so-called bonding pads. In this case, for example, a conductor track of the circuit board may be connected to a bonding pad of the sensor. The bonding pad or the electrode of the sensor, and also the conductive connection of the circuit board, are thereby on the same potential. For example, the bonding pads may have a particularly large thickness, in particular thicker than the electrodes of the sensor. Such thick electrodes made of gold have a particularly smooth and defined surface, such that the bonding pads can be connected to a bonding wire in a particularly simple and reliable manner. In particular, a bonding pad made of gold can be connected particularly easily to a bonding wire made of gold.

The connection socket may in turn be connected to the circuit board via a solder connection, as a result of which the connection socket is able to exchange electrical signals with the sensor and the individual transducers or the A/D converter and/or the A/D converter logic unit.

The printable circuit board may additionally comprise ESD protection diodes. ESD is a harmful electrostatic discharge that may damage both the sensor electronics and the transducers. The protection is in this case already effective against very low voltages of for example 5 V or less.

In the case of a structure with a full bridge, the ESD protection is designed symmetrically, that is to say one diode is provided per part of the full bridge (that is to say a total of 4 diodes) in order to keep the influence of the ESD protection on the full bridge to a minimum, for example to compensate for thermal drift in the diodes or slightly different leakage currents of the diodes.

A connection socket is in particular part of a plug connection or a plug and screw connection or a snap-in connection or a magnetic connection, as a result of which the electrical signals can be routed into a cable, in particular into a multi-strand cable, wherein the cable has a plug corresponding to the socket, such that the electrical signals from the sensor are transferred into the strands of the cable. A magnetic connection in particular has the advantage that it allows a twist-proof connection to the connection socket.

The connection socket and the printable circuit board may be used to implement a voltage supply for the full bridge and/or to read the transverse bridge voltage and/or to read the output signal from the A/D converter and/or to set the measuring mode of the A/D converter logic unit and/or to implement ESD protection.

The connection cable, connected to the electronic components of the printable circuit board via the connection socket, may in particular be used to electrically connect external electronic devices to the sensor. Such an electronic device may in particular be a voltage source that supplies a voltage to the full bridge of the sensor. Such a device may however also be a voltmeter that is able to read the transverse bridge voltage. Such a device may however also be connected to the A/D converter and receive and interpret the digital signals. It is in particular also possible for ESD protection to be implemented via the connection cable. However, it is also possible for a crypto-chip to be connected via the connection cable, which crypto-chip stores production information in order to verify the functionality of the sensor.

The electrical connection may in particular be used to set the A/D converter logic unit such that it is possible to change over between a differential and an absolute measuring mode of the A/D converter. The corresponding measured signals may be tapped off via the electrical connection toward the connection socket, such that the electrical signals that are generated and/or influenced by the transducers are able to be detected and interpreted by the external measuring device.

Since the connection socket provides the electrical signals to external devices, it is not necessary to accommodate all of the circuit electronics on the printable circuit board, as a result of which the sensor device becomes considerably smaller and can be produced cheaper.

In the broader sense, a connection socket may also be an electromagnetic interface via which the signals from the A/D converter are also able to be transported externally via a radio link. By way of example, the sensor device may have a battery apparatus that supplies the full bridge with current or voltage, wherein the measured signal is coded by the A/D converter and then transported to a measuring device, for example via a Bluetooth Low Energy interface, or a WLAN interface, or via an RFID signal.

The connection socket may be a magnetic connection socket.

A magnetic connection socket means that the plug is held in the socket through magnetic forces. This may for example be achieved in that a first magnet is inserted and oriented in the socket, and a second magnet is inserted and oriented in the plug, wherein the mutually facing sides of the magnets each have a different polarity, such that an attractive magnetic force acts between the magnets and accordingly holds the magnetic socket and the magnetic plug together. In this way in particular a twist-proof or polarity reversal-proof connection is realized.

This has the advantage that the sensor device can easily be connected and it is not necessary to use any screw connection or plug connection. The magnetic connection has no mechanical signs of fatigue and allows rapid exchanging of the sensor device. It is additionally possible to ensure a well-defined holding force of the connection due to the magnetic fields. The connection is thus protected against incorrect operation by the user, such as particularly tight or loose fastening.

Properties specific to the housing are described below.

The housing may enclose the sensor and the connection electronics, wherein the housing may have an opening for —connecting the connection electronics and wherein the housing may have a measurement opening, through which at least the deformable parts of the cantilevers of the sensor protrude from the housing.

This may mean that the sensor and the connection electronics lie within the housing, such that the housing shields the connection electronics and the sensor from environmental influences.

This may in particular be electromagnetic radiation, but also an electrostatic discharge, moisture, heat and other effects, in particular mechanical effects.

The housing may in particular be slightly conductive, such that an electrostatic charge can be dissipated. For this purpose, the housing may in particular be connected to a grounding connection on the printable circuit board. The internal components may thereby be protected against ESD damage. Preferably, some or all of the components connected to the printable circuit board may also be grounded.

The opening for the contact connection of the connection electronics is in particular the opening that may be used as a connection socket, such that an electrical connection to the connection electronics is made possible in spite of the housing. The opening may in particular receive the connection cable and thus ensure a mechanically stable connection. The connection may also be such that it allows the sensor device to stand safely on a surface.

The housing also has a measurement opening, through which at least the deformable parts of the cantilevers of the sensor protrude from the housing. It is thereby possible for the cantilevers to interact with the environment and likewise to be given the ability to examine a sample with regard to the analyte.

The openings may be sealed off by rubber seals.

This makes it possible in particular to prevent moisture or fluid from being able to penetrate into the inside of the sensor device or of the housing and for example causing a short circuit. The rubber seals may in particular have a slight conductivity such that ESD protection can be ensured. The seal additionally preferably has no protein adhesion or any other influence on the measurement.

The seals may in particular be suitable for sealing the potentially infectious samples and, together with the sensor, be suitable for safe disposal.

The housing may consist of two parts that are able to be connected to one another by a snap-in connection.

A snap-in connection has the advantage that the housing parts are joined together quickly and preferably lock fixedly to one another. A snap-in connection additionally has the advantage that the connection does not have to be held by screws, meaning that the number of parts of the sensor device is reduced. This for example reduces the costs of the sensor device.

The measurement opening may be surrounded by a thread, wherein the thread preferably corresponds to the thread of a sample vial.

This may mean that a thread is arranged around the measurement opening. A thread may in particular be arranged in a tubular component that is fastened above the measurement opening, wherein the tubular component is preferably locked to the housing when the housing parts are joined together.

A sample vial is a vessel in which the sample can be stored and that is suitable for the safe transport of the sample. The sample vessel may typically have a cap, for example a screw cap, wherein the screw cap has a certain thread diameter and a certain thread pitch. The cap may be used to open and close the sample vial.

The sample vial may in particular be suitable for transporting a virus or for transporting a sample contaminated with an analyte or with a virus. By way of example, a sample vial may be a Copan U™ 359C sample vial.

The thread that surrounds the measurement opening may be a mating thread corresponding to the sample vial, such that the sample vial is able to be screwed to the thread. The thread around the measurement opening may in particular be used to establish a secure connection between the sample vial and the sensor device such that no sample fluid leaks out. The thread may in particular have a thread stop such that it is possible to avoid over screwing and a high mechanical load on the rubber seal.

The sample vial may thus in particular be connected to the sensor device such that the cantilevers can be brought into contact with the sample. It is thereby possible to perform a measurement of the incidence of the analyte or of the virus in the sample without the sample having to be removed from the sample vial. This increases the safety of the sensor device, since there is no handling of a sample fluid that is potentially contaminated.

The housing may have a protective cap for the sensor, in particular for the deformable parts of the reference and test cantilevers, wherein the protective cap protects the deformable parts from a sudden action on the sample, but allows a controlled supply of the sample to the deformable parts of the reference and test cantilevers.

This has the advantage that the sample fluid can be channeled to the cantilever in a defined manner. A protective cap additionally allows a larger structural volume of the sensor, such that a particularly large number of antigens are able to diffuse to the sensor and be detected there. The protective cap is preferably of a size such that microfluidic aspects in the flow behavior of the sample fluid can be ignored. The protective cap may in particular also offer ESD protection.

The protective cap may preferably also have microfluidic properties and support filtering of the sample and/or targeted guidance of the analyte to the cantilevers.

As soon as the sensor device and the sample vial are screwed to one another, the cantilevers are subjected to the mechanical action of the sample. However, this action has to be reduced to an extent such that the sensitive cantilevers are not destroyed by the mechanical influence. For this reason, the housing has a protective cap for the deformable parts of the reference and test cantilevers that extends over the cantilevers in a manner of a shield, such that the sample is braked strongly before it impacts the cantilevers and exerts a lower mechanical force on the cantilevers. A supply of the sample is accordingly already ensured in a controlled manner when a mechanical obstacle is located between the test cantilevers and the sample.

The protective cap in particular also protects the cantilevers against residues from sample removal aids, such as for example cotton swabs that have been used to remove the sample or agitator beads that have been used when preparing the sample. A protective cap may also accordingly be a fine screen or an osmotic layer that allows a controlled supply of the sample fluid to the cantilevers.

For example, for a measurement, the screw connection of the sample vial is opened and the sensor device is screwed onto the sample vial such that the sample is located in the closed part of the sample vial and does not leave the sample vial. In order to connect the sample to the reference and test cantilevers, the sample vial and the sensor device are turned over, that is to say inverted, such that the sample falls in the direction of the cantilevers. Due to the protective cap, the sample does not reach the cantilevers directly, but rather is braked such that any harmful mechanical action on the cantilevers is prevented. In this scenario, the reference and test cantilevers are now located upright underneath the protective cap, while the sample or sample fluid rises along the orientation of the cantilevers, starting from the measurement opening, and comes into contact with the receptor and reference layers. The protective cap must in this case limit the amounts of fluid flowing to the cantilevers only to such an extent that reliable detection is still guaranteed.

The transducers may then detect the deflection of the reference and test cantilevers, wherein the full bridge is used to generate an electrical signal that is converted, for example by an A/D converter, into a digital signal that is then transmitted through the conductor tracks of the printable circuit board to the connection socket, which is then transmitted via a preferably magnetic connection between the connection socket and connection plug into a conductive cable that is connected to a measuring device, wherein the measuring device interprets the received electrical signal.

The sensor device may be connected to an evaluation station that is configured to evaluate the measured signals from the transverse bridge voltage detector and/or the A/D converter.

The evaluation station may in particular be a computer or a smartphone or another device that is capable of interpreting electrical signals. The evaluation station in particular comprises programs that are able to evaluate the measured signals from the transverse bridge voltage detector and/or the A/D converter. These programs may in particular be used to set the measuring mode of the A/D converter logic unit.

The evaluation station may communicate with a computer system, in particular with a smartphone, in a wired or wireless manner, wherein a display of the evaluation is displayed on the computer system.

For example, the evaluation station may be connected to the computer system via a USB cable or an Ethernet cable. However, it is also possible for the evaluation station to communicate with the computer via Bluetooth or WLAN or another radio connection. The computer may in particular also be a smartphone on which a corresponding application for controlling the A/D converter logic unit and for detecting the sensor signals is configured in an executable manner.

The computer system may have a display that displays the result of the evaluation, that is to say in particular whether an analyte or a virus has been found in the sample, or whether this is not the case. The application may in this case display that an analyte or a virus has been found in the sample when a certain threshold value of the transverse bridge voltage is reached. However, the display may also display the magnitude of the incidence of the analyte or of the virus in the sample, such that a quantitative measurement result is provided to the user.

It may in particular be possible to use the computer system to enter the measured results from the sensor device into a database, in particular to forward them to an infection chain tracking application.

### BRIEF DESCRIPTION OF THE FIGURES

Preferred further embodiments of the invention are explained in more detail by way of the following description of the figures, in which:

FIG. 1 shows a schematic illustration of a first embodiment of the sensor;

FIGS. 2A, B, C, D show a schematic illustration of the cantilevers;

FIGS. 3A, B show a schematic illustration of a second embodiment of the sensor;

FIG. 4 shows a schematic illustration of a third embodiment of the sensor;

FIGS. 5A, B, C show further illustrations of further embodiments of the sensor, and also a circuit diagram of a full bridge;

FIG. 6 shows a schematic illustration of a chip with multiple cantilever pairs;

FIG. 7 shows a schematic illustration of the binding of antigens to antibodies;

FIG. 8 shows an exploded drawing of the sensor device; and

FIGS. 9A, B show a schematic illustration of the sensor device in connection with an evaluation station and a computer.

### DETAILED DESCRIPTION OF EMBODIMENTS

Preferred embodiments are described below based on the figures. In this case, elements that are identical, similar or have the same effect are provided with identical reference signs in the various figures and a repeated description of these elements is partly omitted in order to prevent redundancies.

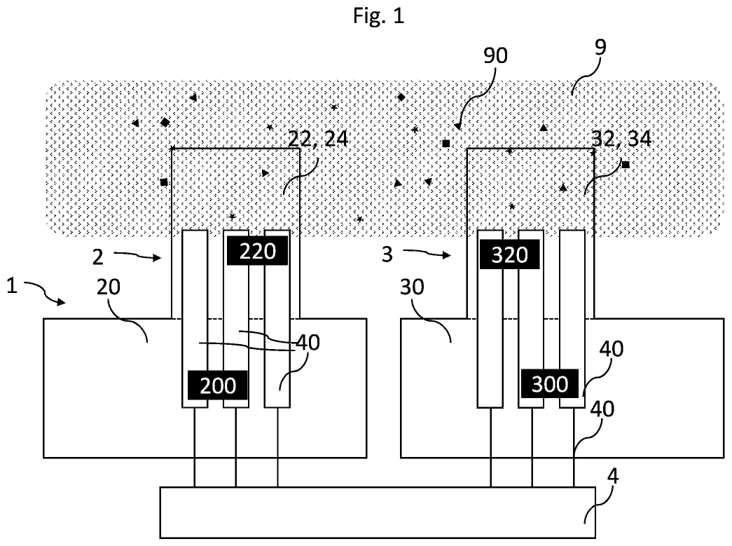

FIG. 1 schematically shows a first embodiment of the sensor 1 according to the invention for converting chemical and/or biochemical information. The sensor 1 comprises a test cantilever 2, which in turn has a base 20, and also a deformable part 22. A passive test transducer 200 is arranged on the base 20, while an active test transducer 220 is arranged on the deformable part 22.

In a similar manner, the sensor 1 also has a reference cantilever 3, which in turn has a base 30 with a passive reference transducer 300, and a deformable part 32 that has an active reference transducer 320.

The transducers 200, 220, 300, 320 are each connected, via electrodes 40, to electronics 4 that are capable of recording or of forwarding a measured signal from the transducers 200, 220, 300, 320, while the electronics 4 are likewise capable of supplying the transducers 200, 220, 300, 320 with current and/or voltage.

The sensor 1 is tasked with indicating the incidence and/or the concentration and/or the amount of an analyte 90 in a sample 9.

In FIG. 1, the sample 9 is a fluid that has been produced by a test subject, for example by treating a swab, in particular a nose swab or a throat swab. However, it may also be the case that the sample 9 is saliva or blood or another bodily fluid. However, it may also be the case that the sample 9 is a gargling fluid that the test subject has gargled. It may also be the case that the sample 9 has been obtained and/or synthesized from a tissue sample or another substance taken from the test subject. The analyte 90 may in this case be dissolved in the sample, or be present in undissolved form, as a suspension or dispersion or emulsion.

In any case, the sensor 1 should be used to examine the sample 9 with regard to the incidence and/or a concentration and/or an amount of an analyte 90 in the sample 9. For this purpose, a receptor layer 24 is applied to the test cantilever 2, with which an analyte 90 can interact, or a receptor layer 24 that is able to adsorb or absorb the analyte 90. In the case of adsorption, the analyte 90 would adhere to the surface of the receptor layer 24, while in the case of absorption the analyte 90 would penetrate into the interior of the reference layer 90.

If the sample 9 contains an analyte 90, this analyte may thus interact with the receptor layer 24. This may cause the surface stress of the portion of the deformable part 22 of the test cantilever 2 coated with the receptor layer 24 to change, resulting in a deformation of the of the deformable part 22 of the test cantilever 2. The active test transducer 220 therefore registers a deformation and/or change in the surface stress of the deformable part of the test cantilever 2, which is in turn interpreted as a measured signal in the electronics 4.

However, a force exerted by the active test transducer 220 may already be registered due to the interaction with the sample fluid 9, for example since only the surface stress of the fluid acts on the deformable part 22 of the test cantilever 2 and bends it. The presence of an analyte 90 is accordingly not responsible for such a deformation.

In order to establish the magnitude of this basic action of the sample 9 on the test cantilever 2, the reference cantilever 3 is also brought into contact with the sample 9 at the same time as the test cantilever 2. For this purpose, the reference cantilever 3 has a reference layer 34, with which receptor layer an analyte 90 can interact, or a reference layer 24 that is not able to adsorb or absorb the analyte 90. In this case, any interaction with the analyte 90 should be avoided in order to allow differentiation with regard to the measured signal from the test cantilever 2.

Since both the test cantilever 2 and the reference cantilever 3 interact with the sample 9, both cantilevers 2, 3 interact similarly with the sample 9. The difference in this case is however that the test cantilever 2 is additionally able to interact with any analyte 90 that is present via its reference layer 24. The measured signals from the active transducers 220, 320 accordingly differ if an analyte 90 occurs in the sample 9. The magnitude of the difference between the measured signals may accordingly, in the simplest case, be used to infer the amount of the incidence of the analyte 90 in the sample 9.

The test cantilever 2 and the reference cantilever 3 however measure the incidence of the analyte 90 in the sample 9 at different positions. There may be different environmental conditions at different positions of the sample, such as for example temperature fluctuations or concentration gradients, etc. These different environmental conditions may be measured by the passive transducers 200, 300.

The passive transducers 200, 300 are arranged on the base and preferably do not detect any measured signal in the event of a deformation of the deformable part 22, 32 of the reference or test cantilevers 2, 3. However, the base level of the measured signal from the passive transducers 200, 300 may be influenced due to these different environmental conditions. Since, for each measured value from the active transducers 220, 230, a comparison value is provided by the passive transducers 200, 300, this comparison value considering the environmental conditions in isolation, the influence of the environmental conditions on the measured signals from the active transducers 220, 320 may be determined and reduced, or factored out or isolated.

The sensor 1 may accordingly be used to analyze the incidence of an analyte 90 in a sample 9 in isolation, since the influence of interactions that are not associated with the analyte 90 are reduced and isolated due to a large number of measurement points on the reference and test cantilevers 3, 2. This allows a high measurement accuracy of the incidence of the analyte 90 in the sample 9.

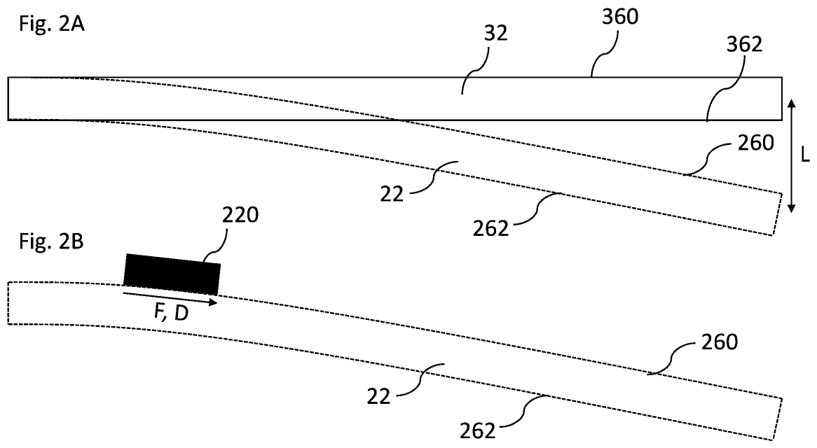

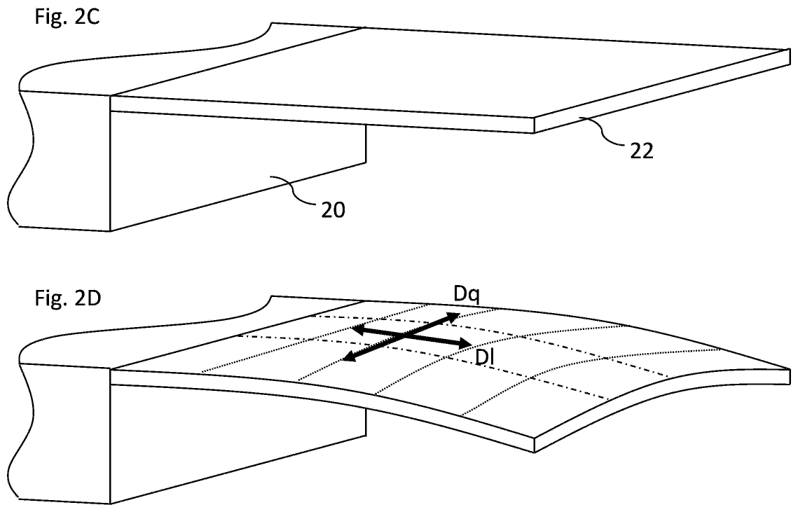

FIG. 2A shows the comparison of the deformable parts 32, 22 of the reference and test cantilevers 3, 2 in the event of a deformation and longitudinal stretching. The deformable part 32 of the reference cantilever 3 has an upper surface 360 and a lower surface 362. The deformable part 22 of the test cantilever 2 likewise has an upper surface 260 and a lower surface 262. If an analyte 90 of the sample 9 interacts with the test cantilever 2, or with the receptor layer 24, the deformable part 22 deforms from the stationary part (that transitions into the base of the test cantilever) toward the freely movable part of the deformable part 22. The deflection L that is shown is in this case given by the relative deflection between the deformable part 32 of the reference cantilever 3 and the deformable part 22 of the test cantilever 2 due to the interaction with the analyte 90.

The deformation of the deformable part 22 of the test cantilever 2 is shown in FIG. 2B. Deformation. The cause of this is that the upper surface 260 and the lower surface 262 expand to different extents. Due to the large stretching D on the upper surface 260, an active transducer 220 applied thereto may register a change in surface stress and/or a stretching force F. The registered change in the surface stress and/or the stretching force F may in this case be converted into an electronic signal by the active transducer 220 or influence an existing electronic signal, for example an applied voltage. This may for example take place by the transducer changing the resistance if it experiences a stretching force F, which in turn results in a stretching of the transducer 220.

The transducer could also detect a contraction of the surface on which it is arranged. In the embodiments shown, the transducers are however always arranged on surfaces for which a stretching is expected.

The stretching and/or change in surface stress and/or force detected by the transducer may however also be a bending force or a shear force or be brought about by a bending force or shear force or generally be based on the modulus of elasticity of the respective cantilever. The attachment of the deformable part 22, 32 to the base 20, 30 in particular results in the deformable part 22, 32 being oriented along a bending curve due to a force exerted by a change in the surface stress of the test cantilever. The resulting bending curve is described in particular by the geometry, in particular the surface moment of inertia of the cantilever, and by the mass of the cantilever and the modulus of elasticity. The bending curves may be described for example in accordance with beam theory.

The different surface stresses on the lower side and the upper side of the cantilever accordingly result in the described deformation or stretching of the cantilever.

Beam theory makes it possible for example to predict the point of the deformable part 22, 32 at which the stretching D will be largest. It is possible to arrange the active transducer 220, 320 at this point in order to achieve an optimum signal-to-noise ratio and in order to react as sensitively as possible to the stretching. When positioning the transducers precisely, however, other boundary conditions should also be taken into consideration.

The orientation of the transducers relative to the orientation of the cantilevers plays a particularly important role. FIG. 2C shows a non-deflected cantilever for example. If the cantilever comes into contact with the analyte, then the surface stress changes and there is a deformation of the material, as shown in FIG. 2D. FIG. 2D illustrates that the cantilever experiences a deformation perpendicular to the base 20, or to the bending edge. This is accompanied by a longitudinal extension DI of the upper surface. At the same time, a deformation takes place parallel to the base 20, or to the bending edge, which is accompanied by a transverse extrention Dq of the upper surface. The geometry of the cantilever makes it possible to define the direction along which a larger extension D is brought about. The transducer may in particular be oriented along this direction in order to generate a particularly large measured signal.

Over-dimensioning a mechanical stretching at the location of the transducer makes it possible to improve the signal ascertained by the transducer even further. Such over-dimensioning may for example be achieved through the arrangement and form of the electrodes.

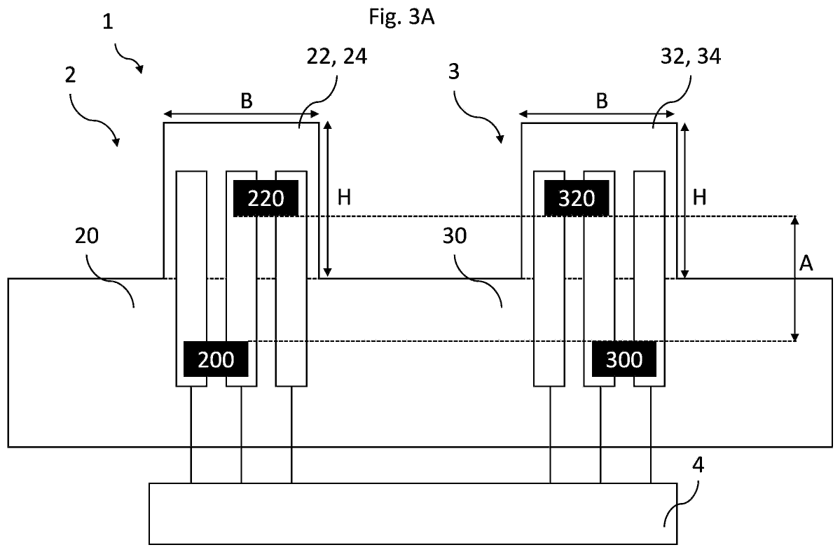

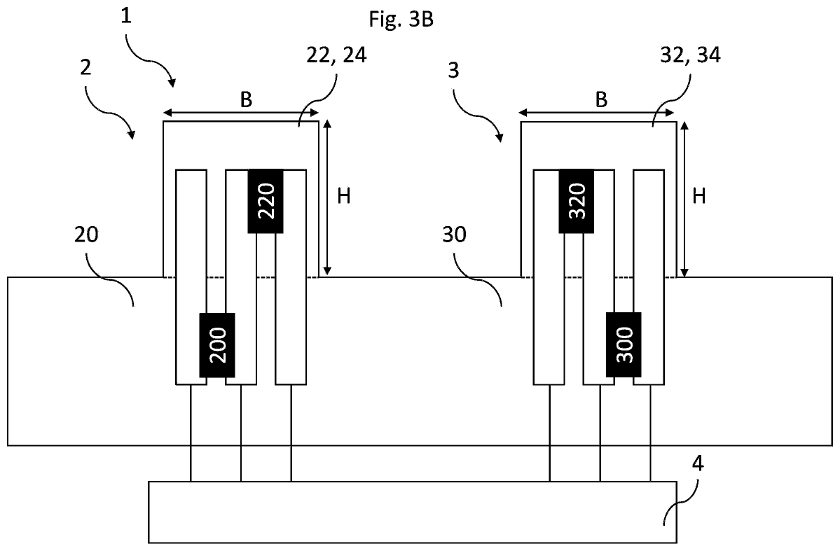

FIG. 3A shows a further embodiment of the sensor 1. The reference cantilever and the test cantilever 2 in particular have identical geometric dimensions; in particular the height, width and thickness of the reference cantilever 3 correspond to the height, width and thickness of the test cantilever 2. An stretching D is thereby generated on the upper surfaces 260, 360. Since the geometrical dimensions of the cantilevers 2, 3 are identical, an identical dependency of the measured signal on the stretching is accordingly also expected.

The width B of the cantilevers is preferably identical to the height H of the cantilevers 2, 3, thereby allowing a particularly large stretching D on the upper surface 260, 30 of the cantilevers 2, 3. For example, the cantilevers are in this case less than 100 μm wide, less than 100 μm long and less than 1 μm thick, in particular 50 μm wide, 50 μm long and 0.3 μm thick.

In the embodiment of the sensor 1 in FIG. 3, the bases 30, 20 of the reference and test cantilever 3, 2 are additionally arranged on the same overall base. There is accordingly a direct mechanical connection and interaction of the cantilevers via the overall base. This makes it possible for example to reduce the various environmental influences on the cantilevers 2, 3, since the cantilevers 2, 3 can be arranged closer to one another. The bases 30, 20 of the reference and test cantilevers 3, 2 may in particular also be formed in one piece with one another. This ensures that the bases also have the same material-specific binding properties, such that the measurement results from the passive and active transducers 200, 220, 300, 320 can be compared well with one another.

The distance A between the active transducers 320, 220 and the passive transducers 300, 200 is measured along the height direction H of the cantilevers. The distance A is in particular less than 100 μm, thereby ensuring that the transducers are arranged as close as possible to one another, such that for example spatial environmental influences on the transducers are reduced.

FIG. 3B shows a further embodiment in which the transducers 200, 220, 300 and 320 are oriented perpendicular to the base 20, 30. While a transverse stretching of the cantilevers 2, 3 is measured along the bending edge in FIG. 3A with the transverse orientation of the transducers, a longitudinal stretching of the cantilevers 2, 3 is measured in FIG. 3B.

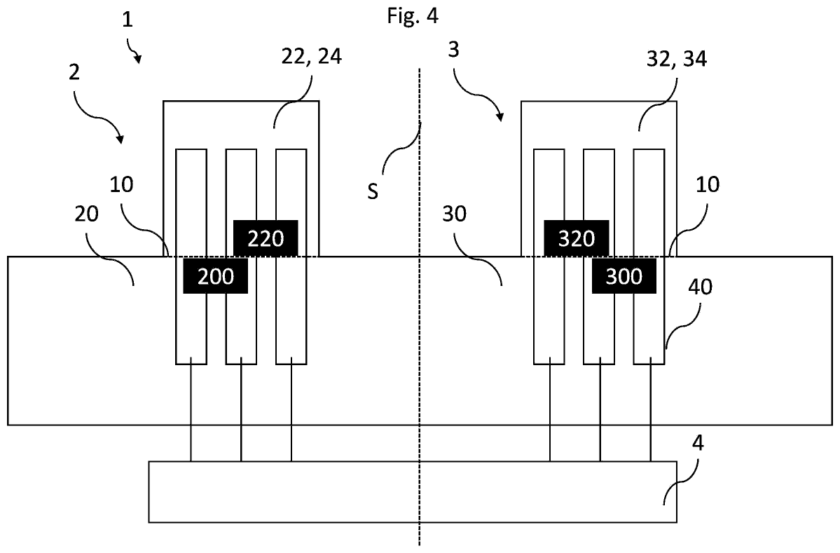

One preferred embodiment in this regard is shown in FIG. 4, in which the active transducers 320, 220 and the passive transducers 300, 200 each lie against the bending edge 10 of the cantilevers 3, 2. Since all of the transducers 320, 300, 220, 200 lie against the bending edge 10, the smallest possible distance A between the transducers 320, 300, 220, 200 is realized. Furthermore, in this embodiment, the electrodes 40 and the transducers 320, 300, 220, 200 are oriented mirror-symmetrically to a mirror axis of symmetry S. The transducers 320, 300, 220, 200 are thus in particular oriented mirror-symmetrically to one another.

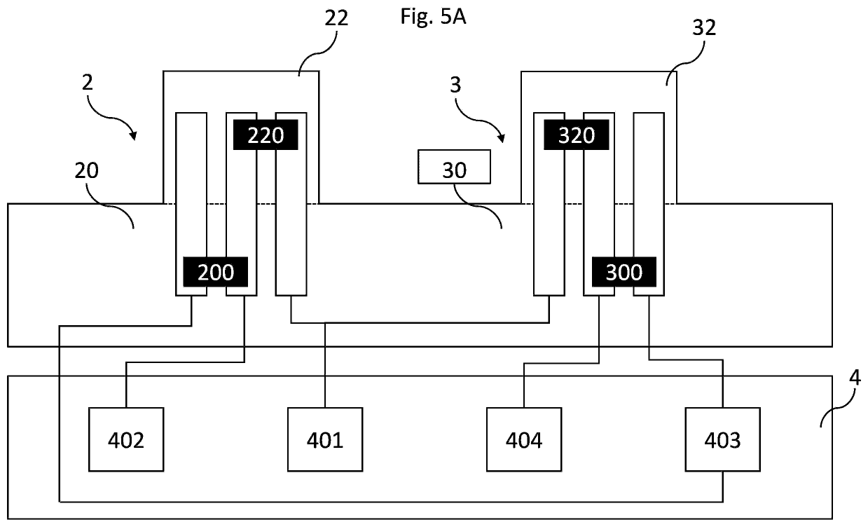

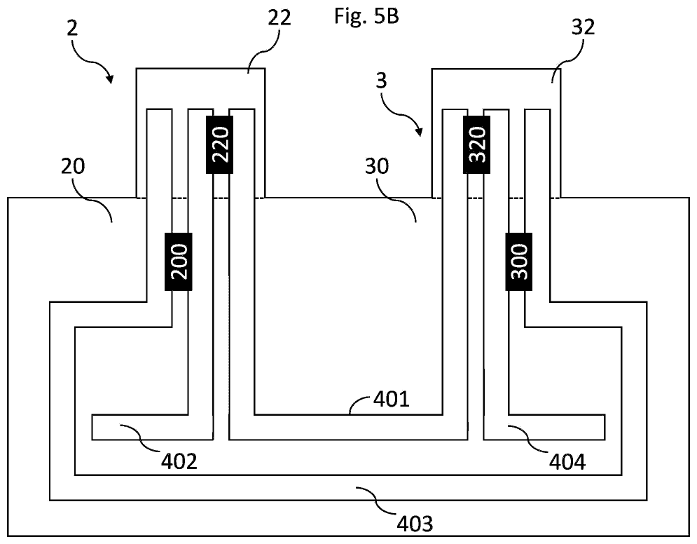

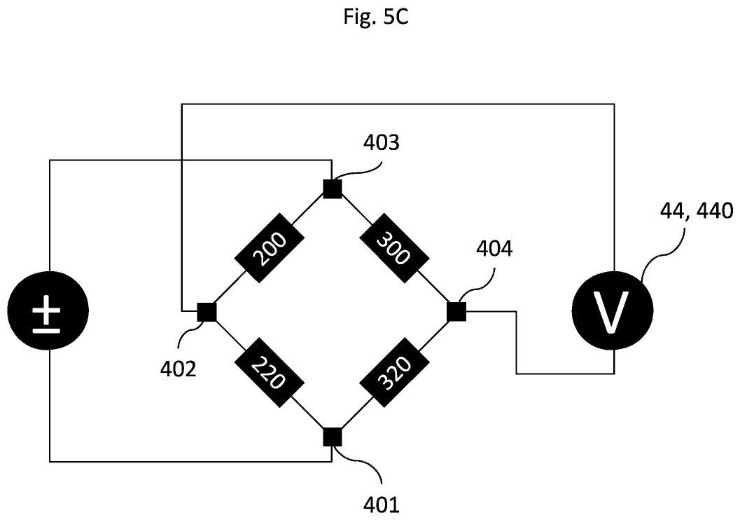

FIG. 5A shows a further embodiment of the sensor 1. The transducers 300, 320, 200, 220 are connected via the electrodes 401, 402, 403, 404. The active transducer 220 is in particular connected to the active transducer 320 via the electrode 401. The passive transducer 200 is furthermore connected to the passive transducer 300 via the electrode 403. The active transducer 220 is additionally connected to the passive transducer 200 via the electrode 402, whereas the active transducer 320 is connected to the passive transducer 300 via the electrode 404. This gives a total of four electrodes via which the transducers are electrically contact-connected to one another. An electrical connection may in this case in particular be achieved by applying the transducers to the electrodes, so as to create a conductive connection. Since the transducers have a thickness, it may in particular be the case that, when electrodes are applied subsequently, no conductive connection to the electrodes would be able achieved at the edges of the transducers. This is only ensured when the thickness of the electrodes is larger than the thickness of the transducers.

FIG. 5B shows a further embodiment of the sensor 1. The electrodes that connect the transducers 200, 220, 300, 320 have an overall mirror-symmetric structure. Currents flow through the electrodes or voltages are present there, such that, when these electrodes have an asymmetric design, there may be asymmetric crosstalk of electrical signals on the other electrodes. This mutual influencing may lead to the generation of a control signal between the electrodes, but this may however be avoided by the symmetrical structure.

The transducers 200, 220, 300, 320 are in particular electrically interconnected in what is known as a full bridge. The circuit of the full bridge is shown in FIG. 5C. In the full bridge, a DC voltage or AC voltage is applied between the electrodes 403, 401. The passive and active transducers act as a voltage divider between these electrodes due to their electrical resistances. A full bridge in the form shown has the advantage that no voltage is built up between the electrodes 402, 404 if the ratio of the resistances of the passive transducer 200 to the active transducer 220 of the test cantilever 2 is identical to the ratio of the resistances of the passive transducer 300 to the active transducer 320 of the reference cantilever 3. The deviation of one resistance is thus in particular sufficient to change the resistance ratios, and thus to build up a voltage between the electrodes 402, 404.

When the reference cantilever 3 and the test cantilever 2 interact with the sample 9 and the analyte 90, then both deformable parts 22, 32 for example experience a change in surface stress, which is larger for the deformable part 22 of the test cantilever 2 than for the deformable part 32 of the reference cantilever 3. The resistance of the active test transducer of the deformable part 22 of the test cantilever 2 will accordingly vary to a larger extent than for the reference transducer 320 of the deformable part 32 of the reference cantilever 3. If the resistances of the passive transducers 200, 300 do not change or at least change identically, a change in the resistance ratios results from the deformation of the deformable part 22 of the test cantilever 2 due to the interaction with the analyte 90 in the sample 9, which interacts specifically with the reference layer 24 of the test cantilever 2. In the event of such an interaction, a voltage builds up between the electrodes 402, 404, such that a force exerted on the active test transducer 220 relative to the active reference transducer 320 can be indicated in the form of a transverse bridge voltage VB. The transverse bridge voltage VB is preferably scaled with the incidence of the analyte 90 in the sample 9, thereby allowing a quantitative assessment of the measured signal.

A transverse bridge voltage detector 44 is able to indicate the transverse bridge voltage VB externally or forward it, such that the fact that a transverse bridge voltage VB is present becomes visible to the user of the sensor 1. Such a transverse bridge voltage detector 44 may in particular also be formed by an A/D converter, wherein the A/D converter converts the transverse bridge voltage VB into a digital signal that may be forwarded to an external measuring device. The A/D converter may in particular be operated in two different measuring modes. The first measuring mode is the differential measuring mode, in which the transverse bridge voltage VB is measured and a relative measured value for the deformation of the two reference and test cantilevers 3, 2 is thus generated. In this differential measuring mode, the measured signals from all of the transducers 200, 22, 300, 23 are taken into consideration, such that the output signal from the A/D converter is a measured signal without environmental influences, through which it is possible to infer the relative deformation of the deformable parts 32, 32 and thus the incidence of an analyte 90.

The second measuring mode is the so-called absolute measuring mode. In the absolute measuring mode, the transverse bridge voltage is not detected, but rather the signals at the electrodes 402 and 404 are tapped off in a manner isolated from one another, such that it is possible to make a statement about the respective deflections of the deformable parts 32, 22. This information remains unavailable to the user in the differential measuring mode.

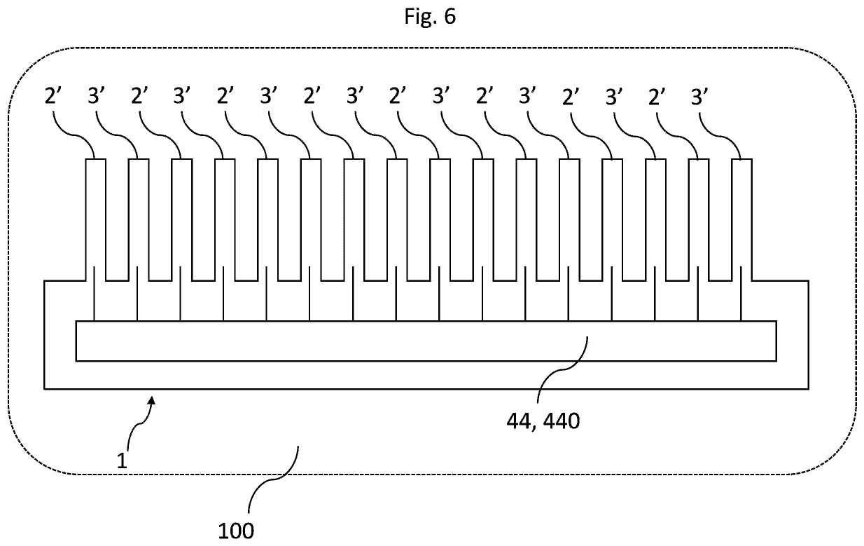

FIG. 6 shows a further embodiment of the sensor 1. The sensor 1 in this case comprises multiple cantilever pairs, wherein here each cantilever pair comprises a reference cantilever 3′ and a test cantilever 2′. The reference cantilever 3′ and test cantilever 2′, or the corresponding transducers, are, as in FIGS. 5A to C, electrically connected to one another via an electrode circuit, such that a transverse bridge voltage VB′ can be tapped off for each cantilever pair. The transverse bridge voltage VB′ may be tapped off from each cantilever pair by the A/D converter 440, or by the transverse bridge voltage detector 44. The measured signal from a specific cantilever pair may in particular for example be output in the A/D converter 440 via an A/D converter logic unit, or the integrated measured signal from all cantilever pairs may be output, or a combination thereof. It is thus in particular possible to average the measured signals over various cantilever pairs, such that the incidence of an analyte 90 is indicated with higher statistical significance. However, it is also possible for various reference and receptor layers 34, 24 to be applied to the various cantilever pairs, such that such a sensor 1 may be used to examine the sample 9 for different analytes 90 at the same time. By way of example, however, it is also possible for a single reference cantilever 3 to serve as reference for multiple test cantilevers 2.

The sensor 1 is in particular embodied with the multiplicity of cantilever pairs on a chip 100. A chip may in this case mean that the sensor 1 has been fabricated from a single substrate, such that for example the various cantilevers 2, 3 are mechanically connected to one another. However, it may also be the case that the chip 100 comprises a further electronic circuit, which is for example a CMOS circuit, that is to say a semiconductor circuit that taps off the transverse bridge voltage VB and directly processes it further. Such a semiconductor circuit in combination with a sensor is also called a system-on-a-chip.

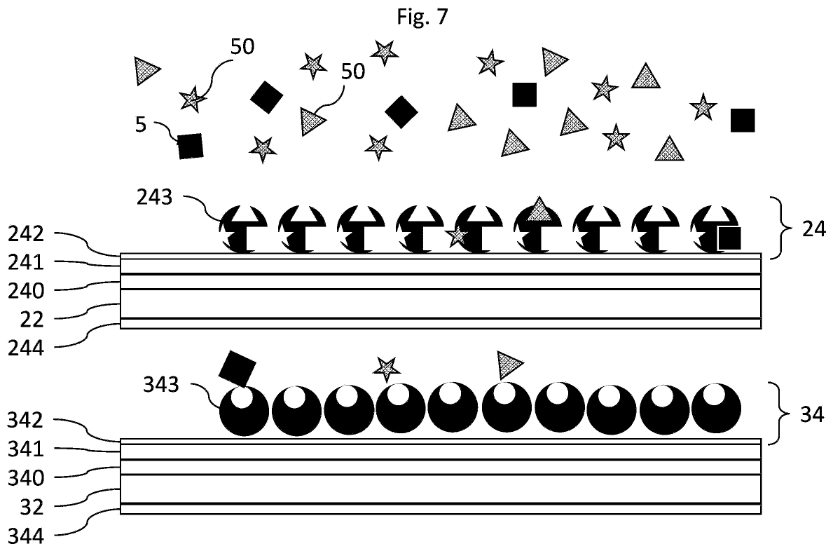

FIG. 7 schematically shows the structure of the various deformable parts 22, 32 of the reference and test cantilevers 3, 2. The structure of the cantilevers is identical apart from the receptor layer and the reference layer, meaning that an interaction with the sample or the surrounding medium, along with the mechanical design of the cantilever, is largely identical.

An activation layer 34, 24 is applied to the deformable part 32, 22 of the reference and test cantilever 3, 2, respectively. An activation layer 240 is configured to promote adhesion between the surface of the deformable part 32, 22 and a further layer 241, 341. The activation layer 240 is furthermore tasked with bringing about an asymmetric layer structure of the cantilever 3, 2, such that there is the greatest possible difference in the stretching of the upper surface of the cantilever and the lower surface of the cantilever. The adhesion promotion layer, or the activation layer 240, may in particular comprise gold or consist of gold.

A so-called self-assembling monolayer 241 may then be applied to the gold layer 240, which can compensate for the surface unevennesses of the gold layer and at the same time provides adhesion promotion for a further layer, specifically the reference and receptor layers 34, 24.

The structure of the reference and receptor layer 34, 24 is different. Both layers are however based on a layer that may comprise so-called protein A 242, which firstly binds to the self-assembling monolayer 241, 341, but also has and is able to bind, on its surface, antibodies 243 or isotype control antibodies 343.

The antibodies 243 are proteins that react to an antigen 5, or bind thereto and thus for example mark virus cells in the human immune system, such that the immune system is accordingly able to destroy the marked virus in order for example to stem or to prevent a viral outbreak. The antibodies 243 are largely specific to the antigen 5, but may however also interact with other similar antigens 50. FIG. 7 shows that the antibody 243 can interact with the antigen 5 and the similar antigens 50 to some extent.

In contrast to the antibody 243, the isotype control antibody 343 is a protein that preferably does not interact with the antigen 5 in an ultra-high-specific manner. This makes it possible to virtually rule out any interaction with a specific antigen 5. This is shown in FIG. 7 by the fact that the isotype control antibody 343 can interact only with two similar antigens 50, but not with the one shown schematically as a square here.

Since the test cantilever 2 has an antibody 243 and the reference cantilever 3 has an isotype control antibody 343, it is ensured that, in the sample 9, the analyte 90, if the analyte 90 is an antigen 5, can interact only with the test cantilever 2. This ensures that the relative deformation, brought about by the analyte, of the test cantilever 2, in comparison with the deformation of the reference cantilever 3, is based only of the presence of the analyte 90 or of the antigen 5. It is accordingly possible to use this sensor 1 to detect an antigen 5 reliably and quickly.

In contrast to the upper surface of the cantilevers, the lower surface of the cantilevers is passivated. Such passivation may lead to an interaction, or binding, or absorption or adsorption of an analyte 90 in the sample 9 in or on the cantilevers being avoided. However, such a passivation layer in particular also contributes to increasing the asymmetry of the layer structure in order to bring about the largest possible stretching effect on the upper surface of the cantilever 3, 2. The passivation layer may in particular comprise trimethoxysilane and/or a blocking substance.

The sensor that is shown may in particular be used to detect the antigens 5 of a Sars-CoV2 virus or of another virus. For this purpose, the receptor layer 24 of the test cantilever 2 for example comprises Sars-CoV2 antibodies, while the reference layer 34 comprises Sars-CoV2-specific isotype control antibodies. A measured signal is accordingly produced by the sensor 1 when the antigens 5 of a Sars-CoV2 virus are present in the sample 9 and accumulate on the test cantilever 2 or the receptor layer 24.

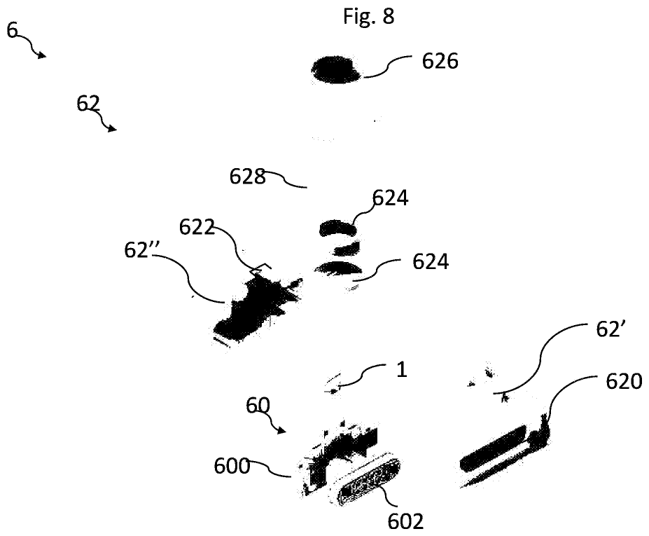

FIG. 8 schematically illustrates the sensor device 6 in an exploded drawing. The sensor device 6 in this case comprises a sensor housing 62, the connection electronics 60 and the sensor 1.

The connection electronics 60 in particular have a printable circuit board 600 that enables the electrical communication between the connection socket 602 and the sensor 1. For this purpose, conductor tracks may be provided on the printable circuit board, these being produced for example from a conductive layer of the printable circuit board in an etching process, or by writing the conductor tracks to the substrate of the printable circuit board in a writing process. The conductor tracks are connected to the electrodes 40 of the sensor 1 at one end, for example by virtue of an electrically conductive wire being bonded both to the conductor tracks and to the electrodes 40. At the other end, the conductor tracks may for example be soldered to the connection pins of the connection socket 602. The printable circuit board 600 thus takes on at least the role of promoting the electrical conduction between the connection socket 602, which has macroscopic dimensions, and the electrodes 40 and the transducer of the sensor 1, which may have microscopic dimensions.

In order to ensure ESD protection, the parts of the sensor device 6 may be slightly conductive, for example have a resistance of less than 1 GΩ, and be grounded via an ESD protection contact on the printable circuit board 600.

The printable circuit board 600 may additionally also have a crypto-chip on which production parameters are stored. This makes it possible to achieve reliable and correct detection of the analyte in the sample fluid.

The connection socket 602 has eight connection pins in FIG. 8. For example, two pins of these may allow electrical conduction from a current source or a voltage source to the full bridge. By way of example, two further pins may directly tap off the transverse bridge voltage VB and provide it to a voltmeter connected via the connection socket 602. For example, two further connection pins may tap off the signal from the A/D converter 44, while two further pins may be used to communicate with the A/D converter logic unit. However, it is also possible for the connection pins to have another assignment.

For example, the connection socket 602 may thus also be used to set the measuring mode of the A/D converter logic unit 440.

It may in particular be the case that the connection socket 602 is a magnetic connection socket. For this purpose, the connection socket may contain magnets the polarity of which, on the connection side of the connection socket 602, corresponds to the reverse polarity of the connection magnets in the connection side of the socket of the plug.

The exploded drawing furthermore shows that the housing 62 consists of two parts 62′, 62″. The two parts of the housing may be connected to one another via a snap-in connection and thereby enclose the connection electronics 60, such that these are protected for example against mechanical shocks, against moisture or against electromagnetic radiation. The housing may in particular be slightly conductive in order to ensure ESD protection for the components inside it. In order to allow a connection of measuring devices and voltage sources to the connection socket 602, and thus indirectly to the sensor 1, one part 62′ has an opening 620, such that the connection socket 602 is externally accessible. The opening 620 may in particular provide a mechanically stabilizing effect for the connection cable.

The housing 62 in particular also has a measurement opening 622 through which at least the deformable parts 32, 22 of the cantilevers 3, 2 protrude externally. This ensures that the cantilevers 3, 2 are also able to interact with the environment, in particular with the sample 9.

In order to enable further sealing of the sensor device 6, the housing 62 is sealed off with rubber seals 624. For example, it is also possible to seal off the housing with only one rubber seal 624 that is embedded in the thread 626. The rubber seals 624 are in particular arranged on the housing side of the measurement opening 622, such that no sample fluid penetrates into the housing 62 of the sensor device 6 so as for example to trigger a short circuit on the printable circuit board 600. A thread 626 may be arranged around the measurement opening 622, wherein the thread 626 corresponds to the thread of a sample vial 92, such that sample fluid is able to be supplied to the cantilevers 3, 2. The thread may in particular have a thread stop in order to avoid overscrewing of the thread. In FIG. 8, the thread is arranged in a cylindrical component that can be jointly enclosed and fastened in a durable manner when the parts of the housing 62′, 62″ are connected.

A sample vial may finally be screwed into this thread 626. In order to protect the cantilevers 3, 2 against any direct mechanical action of the sample 9, the housing 62 has a protective cap 628, which both protects the cantilevers 3, 2 from the direct mechanical action of the sample, but also ensures a controlled supply of the sample 9 to the deformable parts 32, 22 of the reference and test cantilevers 3, 2.

If the sample vial 92 is connected to the housing 62 and the sample vial 92 is turned with the housing 62 such that the sample 9 falls in the direction of the sensor 1, the protective cap 628 acts as a protective shield. However, a controlled supply of the sample 9 may be achieved through a subsequent rise in the level of the sample fluid in the direction of the deformable parts 32, 22 of the cantilevers 3, 2. It is thus possible for the cantilevers 3, 2 to detect the accumulation of an analyte, or of a virus, for a transverse bridge voltage VB to arise across the full bridge, for this transverse bridge voltage VB to be routed to the connection socket 602 via the printable circuit board and to be transmitted from there by a connection plug and a downstream cable to an evaluation station 7 in which the measured signals can be interpreted.

The sample vial 92 may in this case be embodied for a specific sample volume, such that the cantilevers 3, 2 are underneath the sample fluid surface and are covered completely with sample fluid.

The sample vial 92 may in particular be held in the housing 62 such that no potentially infectious sample fluid is able to escape.

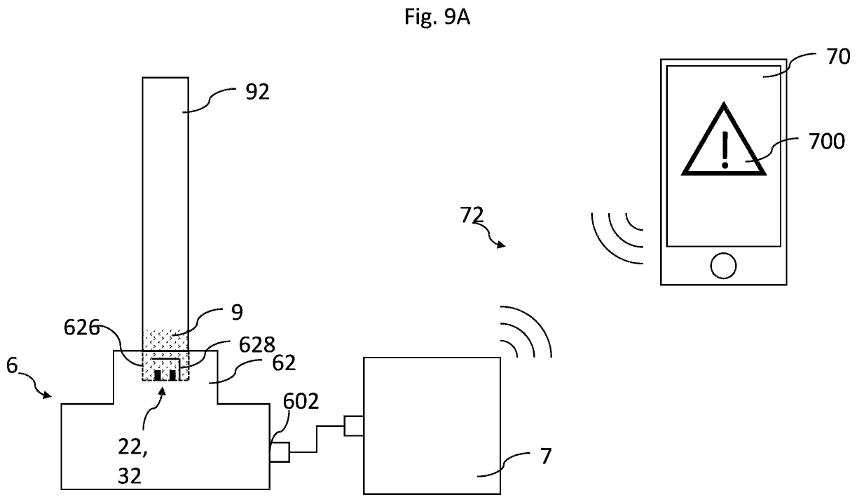

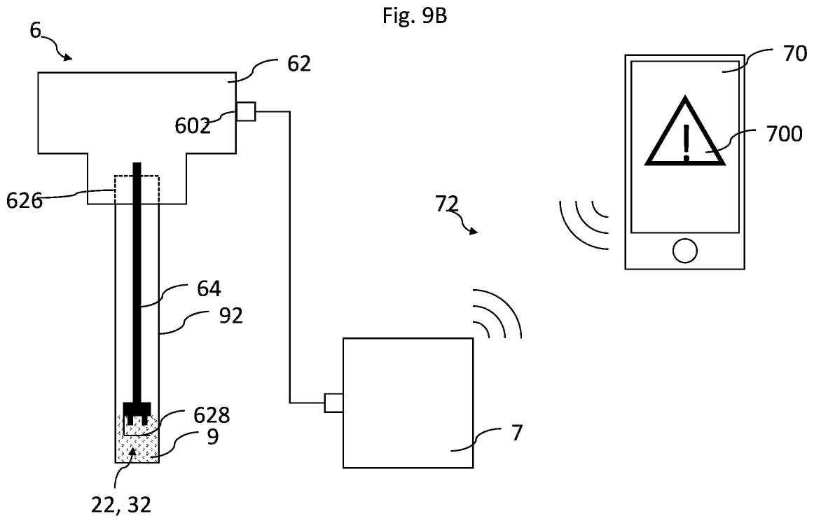

FIG. 9A shows a sensor device 6 that is connected to a sample vial 92 via a thread 626 with a thread stop. The sample vial 92 is in particular closed at the top and open on the thread side, such that the sample 9 is able to reach at least the deformable parts 32, 22 of the reference and test cantilevers 3, 2. Any direct mechanical interaction is in this case prevented by the protective cap 628, but a controlled supply of the sample to the cantilevers 3, 2 and a subsequent mechanical interaction is made possible.

The measured signals produced by the transducers of the cantilevers 3, 2 in the full bridge are transmitted to an evaluation station 7 via the magnetic connection socket 602 and via a cable. The magnetic connection socket 602 may in particular be used to create a twist-proof and polarity reversal-proof connection. ESD protection may furthermore be ensured by the connection cable, by virtue for example of contact-connecting the grounding connection of the printable circuit board.

The evaluation station 7 is configured to evaluate and to interpret the measured signals, for example the transverse bridge voltage VB and/or the digital signals from the A/D converter 44. For this purpose, the evaluation station 7 may in particular comprise a memory, comprise a processor, and comprise communication interfaces, such that the evaluation station 7 is capable of processing and outputting data. The evaluation station 7 may also verify the functionality of the sensor using a crypto-chip installed therein. It is additionally also possible to incorporate the connection cable into the verification process using a crypto-chip in order to ensure reliable and correct analysis of the sample fluid.

The evaluation station 7 may in particular communicate with a computer system 70, in particular with a smartphone, via a wired or wireless interface. For example, the interface that is shown may be a Bluetooth interface or a WLAN interface or an interface based on microwaves (RFID) or magnetic fields (NFC). The evaluation of the measured signal from the evaluation station 7 may finally be displayed on the computer system 70, such that a user is for example made aware of a contaminated sample, that is to say a sample 9 that contains an analyte 90 or a virus 902.

The computer system 70 may in particular also have an interface that is compatible with infection chain tracking programs. A possible test result may in particular be uploaded to a database in order to allow further tracking of the infection chains.

FIG. 9B shows an alternative embodiment of the sensor device 6. In this case, the sensor 1, or the deformable parts 23, 22 of the cantilevers 3, 2, are guided in the direction of the sample 9 via a dipstick 64, wherein the protective cap 628 once again protects the cantilevers 3, 2 against sudden interaction with the sample 9. The dipstick 64 is part of the housing 62, meaning that the measurement opening 622 is located at the end of the dipstick 64. The dipstick 64 may furthermore comprise part of the printable circuit board 600, such that the sensor 1 is able to be arranged at the end of the dipstick 64.

If applicable, all individual features that are illustrated in the exemplary embodiments may be combined with one another and/or exchanged without departing from the scope of the invention.

### LIST OF REFERENCE SIGNS

1 Sensor
10 Bending edge
2 Test cantilever
20 Base
200 Passive test transducer
22 Deformable part
220 Active test transducer
24 Receptor layer
240 Activation layer
241 Self-assembling monolayer
242 Protein A
243 Antibody
244 Passivation layer
26 Surface
260 Upper surface
262 Lower surface
3 Reference cantilever
30 Base
300 Passive reference transducer
32 Deformable part
320 Active reference transducer
34 Reference layer
340 Activation layer
341 Self-assembling monolayer
342 Protein A
343 Isotype control antibody
344 Passivation layer
36 Surface
360 Upper surface
362 Lower surface
4 Electronics
40 Electrode
400, 401, 402, 403 Electrodes
42 Transverse bridge voltage detector
44 A/D converter
440 A/D converter logic unit
6 Sensor device
60 Connection electronics
600 Printable circuit board
602 Connection socket
62 Housing
620 Opening
622 Measurement opening
624 Rubber seal
626 Thread
628 Protective cap
64 Dipstick
7 Evaluation station
70 Smartphone
700 Display
72 Wireless connection
9 Sample
90 Analyte
900 Antigen
902 Virus
92 Sample container
F Force
L Deflection
D Stretching
AT Distance between active and passive transducer
AE Distance between electrodes
S Axis of symmetry
VB Transverse bridge voltage

What is claimed:

1. A sensor device for detecting at least one of an incidence, a concentration and an amount of an analyte in a sample, the sensor device comprising:
a sensor configured to convert at least one of chemical and biochemical information of an analyte in a sample into an electrical signal;
connection electronics; and
a housing,
wherein the sensor comprises a test cantilever that has a base and a deformable part, wherein a receptor layer for selective reception of the analyte is applied at least to the deformable part of the test cantilever, and
wherein the sensor further comprises a reference cantilever that has a base and a deformable part, wherein a reference layer for selective non-reception of the analyte is applied to the deformable part of the reference cantilever.

2. The sensor device according to claim 1, further comprising:
a passive test transducer arranged on the base of the test cantilever and an active test transducer arranged on the deformable part of the test cantilever; and
a passive reference transducer arranged on the base of the reference cantilever and an active reference transducer arranged on the deformable part of the reference cantilever,
wherein the respective active and passive reference transducers are configured to output an electrical signal corresponding to the at least one of the incidence, concentration and the amount of the analyte in the sample.

3. The sensor device according to claim 2, wherein the selective reception of the analyte by the receptor layer and the selective non-reception of the analyte by the reference layer causes at least one of a deformation and a change in a surface stress of the test cantilever with respect to the reference cantilever, and the at least one of the incidence, the concentration and the amount of the analyte is inferred by comparing forces detected by the respective transducers or by comparing surface stresses detected by the respective transducers.

4. The sensor device according to claim 1,
wherein the deformable parts of the reference and test cantilevers have identical geometric dimensions,
wherein a width of the deformable parts of the reference and test cantilevers corresponds to a length of the deformable parts of the reference and test cantilevers, and
wherein the bases of the reference and test cantilevers are arranged on a same overall base and the bases are formed in one piece with one another.

5. The sensor device according to claim 2, wherein the respective transducers are electrically interconnected in a full bridge that is configured to build up a transverse bridge voltage (VB) based on electrical properties of the respective transducers.

6. The sensor device according to claim 5, further comprising an A/D converter configured to convert the transverse bridge voltage (VB) into a digital signal and that is configured to be operated in at least one of a differential measuring mode and in an absolute measuring mode using an A/D converter logic unit.

7. The sensor device according to claim 1, further comprising:
a passivation layer applied to lower surfaces of the reference and test cantilevers;
an activation layer applied to upper surfaces of the reference and test cantilevers;
a self-assembling monolayer applied to the activation layer; and
at least one of a reference layer or receptor layer applied to the self-assembling monolayer of the reference and test cantilever, respectively,
wherein the receptor layer comprises antibodies for an antigen and the reference layer comprises an antigen-specific isotype control antibody according to the antibody of the receptor layer.

8. The sensor device according to claim 1, wherein:
the receptor layer comprises single-strand DNA (ssDNA) and/or other DNA fragments that bind specifically to DNA fragments in the sample and the reference layer comprises single-strand DNA and/or other DNA fragments that do not bind to any chemical and/or biochemical and/or physical species in the sample, but correspond to the receptor layer in terms of characteristic parameters, or
the receptor layer comprises single-strand RNA and/or other RNA fragments that bind specifically to RNA fragments in the sample and the reference layer comprises single-strand RNA and/or other RNA fragments that do not bind to any chemical and/or biochemical and/or physical species in the sample, but correspond to the receptor layer in terms of characteristic parameters, or
the receptor layer comprises antibodies and/or other and/or further proteins that are able to specifically bind target proteins and the reference layer comprises specific isotype control antibodies and/or other and/or further proteins that do not bind to any chemical and/or biochemical and/or physical species in the sample, or
the receptor layer comprises scFv antibodies and the reference layer comprises scFv antibody-specific isotype control antibodies; or
the receptor layer comprises Sars-CoV2 antibodies and the reference layer comprises Sars-CoV2-specific isotype control antibodies; or
the receptor layer and the reference layer comprise hydrogels.

9. The sensor device according to claim 6, wherein the connection electronics comprise a printable circuit board that is configured to ensure the electrical communication between a connection socket and the sensor.

10. The sensor device according to claim 9, wherein the connection socket and the printable circuit board are configured to perform at least one of:
implementing a voltage supply for the full bridge,
reading the transverse bridge voltage (VB),
reading the output signal from the A/D converter, setting the measuring mode of the A/D converter logic unit, and
implementing ESD protection.

11. The sensor device according to claim 9, wherein the connection socket is a magnetic connection socket.

12. The sensor device according to claim 1, wherein:
the housing encloses the sensor and the connection electronics,
the housing has an opening for connecting the connection electronics, and
the housing has a measurement opening through which at least the deformable parts of the cantilevers of the sensor protrude from the housing.

13. The sensor device according to claim 12, wherein the openings are sealed by rubber seals.

14. The sensor device according to claim 12, wherein the housing comprises two parts that are connected to one another by a snap-in connection.

15. The sensor device according to claim 12, wherein the measurement opening is surrounded by a thread that corresponds to a thread of a sample vial.

16. The sensor device according to claim 12, wherein the housing has a protective cap for the deformable parts of the reference and test cantilevers, with the protective cap configured to protect the deformable parts against any direct mechanical action of the sample and allows a controlled supply of the sample to the deformable parts of the reference and test cantilevers.

17. The sensor device according to claim 9, wherein the printable circuit board has an ESD protection contact or grounding contact, at least part of the sensor housing has a conductivity of less than 1 GΩ and the at least one conductive housing part is electrically conductively connected to the printable circuit board.

18. The sensor device according to claim 6, wherein:
the sensor device is connected to an evaluation station that is configured to evaluate the measured signals from at least one of the transverse bridge voltage detector and the A/D converter, and
the evaluation station is configured to communicate with a computer system in a wired or wireless manner, wherein a display of the evaluation is displayed on the computer system.

19. The sensor device according to claim 3, wherein at least one of the deformation and the change in the surface stress is achieved in a transverse direction of at least one of the test cantilever and the reference cantilever, with the transverse direction running parallel to the base of the test cantilever and/or of the reference cantilever.

20. The sensor device according to claim 3, wherein at least one of the deformation and the change in the surface stress is achieved in a longitudinal direction of at least one of the test cantilever and the reference cantilever, wherein the longitudinal direction runs perpendicular to the base of the test cantilever and/or the reference cantilever.
